(19) United States
(12) Patent Application Publication
(10) Pub. No.: US 2018/0235992 A1
(43) Pub. Date: Aug. 23, 2018

(54) IMMUNOTHERAPIES FOR MALIGNANT, NEURODEGENERATIVE AND DEMYELINATING DISEASES BY THE USE OF TARGETED NANOCARRIERS

(71) Applicants: RODOS BIOTARGET GMBH, Hannover (DE); AUGUSTUS BIOTARGET, INC., North Myrtle Beach, SC (US)

(72) Inventors: Michael J. SCOLARO, North Myrtle Beach, SC (US); Sean M. SULLIVAN, Escondido, CA (US); Robert K. GIESELER, Werdohl (DE); Constantin HOZSA, Hannover (DE); Marcus FURCH, Frankfurt am Main (DE)

(73) Assignees: RODOS BIOTARGET GMBH, Hannover (DE); AUGUSTUS BIOTARGET, INC., North Myrtle Beach, SC (US)

(21) Appl. No.: 15/747,642

(22) PCT Filed: Jul. 27, 2016
(86) PCT No.: PCT/EP2016/067931

§ 371 Date: Jan. 25, 2018

Related U.S. Application Data
(60) Provisional application No. 62/282,101, filed on Jul. 27, 2015.

(51) Int. Cl. A61K 31/7068; A61K 31/337; A61K 31/12; A61K 47/54; A61K 47/69

(52) U.S. Cl. CPC A61K 31/7068; A61K 31/337; A61K 47/6911; A61K 47/549; A61K 31/12

(57) ABSTRACT

A method for the targeted delivery of the active generic antiproliferative and anti-inflammatory agents gemcitabine, paclitaxel and/or curcumin preferentially or exclusively to antigen-presenting cells (APCs) of the immune system by means of encapsulation into a lipid-based nanocarrier, the CLR-TargoSphere, which is surface-labeled with a Fucose-derivative ligand that exclusively targets C-type lectin receptors (CLRs) on APCs to deliver the active agents intracellularly to myeloid dendritic cells (mDCs), circulating monocytes, macrophages, and tumor-associated macrophages (TAMs) as well as cytotoxic T lymphocytes (CTLs).

### FIELD OF THE INVENTION

The present invention relates to the targeted delivery of the active generic antiproliferative and anti-inflammatory agents gemcitabine, paclitaxel and/or curcumin preferentially or exclusively to antigen-presenting cells (APCs) of the immune system by means of encapsulation into a lipid-based nanocarrier, the CLR-TargoSphere, which is surface-labeled with a Fucose-derivative ligand that exclusively targets C-type lectin receptors (CLRs) on APCs to deliver the active agents intracellularly to myeloid dendritic cells (mDCs), circulating monocytes, macrophages, and tumor-associated macrophages (TAMs) as well as cytotoxic T lymphocytes (CTLs).

### BACKGROUND OF THE INVENTION

### Cancers:

Cancers are the second leading worldwide cause of death, ranking behind only cardiovascular diseases. Among the various traditional approaches to treating cancers, chemotherapy remains the leading treatment option, along with radiation and surgical interventions. However, aggressive chemotherapeutic treatments are associated with toxicity to healthy bystander cells, poor tolerance to effective antineoplastic dosages, and limited treatment success due to the development of multidrug-resistant tumors.

These factors have called for the development of safe and effective targeted treatments. Perez-Herrero and Fernandez-Medarde recently reviewed targeted strategies that allow specific delivery of chemotherapeutic agents to tumors to avoid systemic toxicity as well as toxicity to healthy bystander cells, protect drugs from rapid degradation, increase the half-life and solubility, and also reduce renal clearance. In 2012, approximately 20 tumor antigen-specific antibodies had received approval for the treatment of cancers from different regulatory authorities throughout the world (1).

Depending on safety considerations, some of these antibodies as well as some of the natural ligands of cancer-associated receptors may be employed as the targeting moieties of nanomedicines to target the central nervous system (CNS). However, in order to enable entry of encapsulated pharmaceutical agents into the CNS, it is necessary that the targeted drug delivery system efficiently crosses the blood-brain barrier (BBB) (2). The BBB acts as a physical barrier under normal conditions when there is no evidence of inflammation in the brain endothelium which ordinarily prevents free entry of blood-derived substances, including those intended for therapeutic applications.

### Neurodegenerative and Demyelinating Diseases:

Of all the neurodegenerative diseases, Alzheimer's disease (AD) is the 6^{th} leading cause of death, with a markedly growing proportion of the population suffering from dementia globally. By 2025, the number of US citizens with AD is expected to grow to reach 7.1 million (a 40% increase from 2015).

Delivery of therapeutics into the brain has been a major barrier to the effective treatment of neurodegenerative diseases. This may be achieved by applying targeted drug delivery strategies to ferry therapeutic agents across the blood-brain barrier (BBB) for neoplasms of the brain, via receptor-mediated transcytosis. In this process, the nanocarrier-drug system is transported transcellularly across the brain endothelium, from the blood to the brain interface. This may be achieved by coupling a native receptor to the delivery system (3, 4). However, the treatment of AD has been met with consistent failures.

Although cognitive impairments, loss of executive functions, and progressive dementia seen in AD is believed to be associated with abnormal protein tau and the development of neurofibrillary tangles, as well as increased aggregation of amyloid 13, both triggering the toxic events that lead to progressive neurodegeneration, no drug candidates targeting either the abnormal protein tau or the amyloid cascade have yet produced a successful treatment.

A major limitation in the use of potentially effective therapeutic immune-enhancing and anti-inflammatory agents such as paclitaxel and curcumin for the treatment of AD has been the inability to cross the BBB. This can now be successfully overcome by targeted transportation of the antiproliferative and anti-inflammatory agents across the BBB by migrating APCs.

Novel Nanomedicines have an Immense Potential for Significantly Improving Cancers, Neurodegenerative, and Demyelinating Diseases:

Nanoconstructs such as liposomes are widely used in clinics, while polymer micelles are in advanced phases of clinical trials in several countries. Innovative nanomedicines involve the functionalization of these constructs with moieties that enhance site-specific delivery and tailored release (5, 6).

In the past years, receptor-mediated tumor targeting has received major attention as it improves the pharmacokinetics of various drugs and protects against systemic toxicity and adverse effects that result from the non-selective nature of most current cancer therapeutic agents (7).

Specific receptors allowing for uptake of a drug-loaded targeted nanocarrier include, but are not limited to tumor-associated antigens categorized as (i) hematopoietic differentiation antigens (CD20, CD30, CD33, and CD52); (ii) cell surface differentiation antigens (various glycoproteins and carbohydrates); (iii) growth factor receptors (CEA, EGFR/ErbB1, HER2/ErbB2, c-MET/HGFR, IGFR1, EphA3, TRAIL-R1, TRAIL-R2, RANKL; (iv) vascular targets (VEGFR, αVβ3, α5β1) (8,9).

To date, two polymer-protein conjugates, five liposomal formulations, and one polymeric nanoparticle are approved for clinical use, and due to the clinical advantages of these new targeted treatments, numerous additional clinical trials are currently in progress (10).

Of all the targeted strategies developed, liposome encapsulation has been consistently approved by the FDA for the treatment of cancer. It has been well demonstrated that the use of liposomes for the treatment of solid tumors protects the encapsulated drug from rapid inactivation following parenteral administration and reduces toxicity to healthy tissues before it reaches its site of action (11, 12).

### 1. Gemcitabine Liposomal Formulations for the Treatment of Cancers:

Gemcitabine-loaded PEGylated liposomes studied in vivo protected gemcitabine from enzymatic degradation with improved accumulation in tumor tissues due to increased vascular permeability. Encapsulation increased the half-life of gemcitabine and enhanced its antitumor activity (13, 14).

Gemcitabine prodrug encapsulated in a liposome was reported by Brusa, P. et al. (15).

Additionally, a multidrug liposomal carrier, encapsulating both gemcitabine and paclitaxel has been successfully developed to obtain a synergistic therapeutic effect based on the fact that each compound induces apoptosis by different mechanisms (16, 17).

A nanoparticle drug delivery combining gemcitabine with curcumin has been shown to retard tumor growth, abolish systemic metastases, reduce activation of NF-κB, and reduce expression of matrix metalloproteinase-9 and cyclin D1 in a pancreatic xenograft model, as compared to either drug alone (18,19,20).

A nanoparticle drug delivery conjugate of gemcitabine and paclitaxel was reported by Aryal, S., et al. (21).

### 2. Paclitaxel Modified Formulations for the Treatment of Cancers:

Paclitaxel is a chemotherapeutic agent whose action as a microtubule stabilizer interferes with the normal breakdown of intracellular microtubules during cell division, resulting in apoptosis (programmed cell death) of the cancer cell. Interestingly, it has been shown also to act indirectly upon the immune system by enhancing the presence and number of tumoricidal (M1) macrophages at the tumor site, thereby reducing cancer invasion and metastasis (22, 23).

Paclitaxel is among the third highest prescribed chemotherapy agents globally, approved for many cancers including Kaposi's sarcoma, non-small cell lung cancer, breast, and ovarian cancer. Despite formulations which attempt to target the tumor and avoid systemic circulation, it continues to be associated with therapeutic failures due to the development of tumor resistance and the continued incidence of serious systemic toxicities to bone marrow and normal cell populations.

From a clinical perspective, the original formulation of paclitaxel dissolved in Cremophor (an excipient now termed Kolliphor, a version of polyethoxylated castor oil) is associated with severe toxic and hypersensitive reactions. Cremophor was required to solubilize the drug for intravenous administration. Consequently, many approaches have been developed to administer it systemically to avoid this toxic effect.

One such development of an alternative formulation, is albumin-bound paclitaxel, nab-paclitaxel (Abraxane/Celgene), in which paclitaxel is bound to albumin as an alternative delivery agent. It was approved by the FDA in 2005.

Other formulations have been developed with fewer side effects and improved uptake by cancer cells. These include: DHA paclitaxel (Protarga) in which a fatty acid easily taken up by tumor cells is linked to paclitaxel; PG-paclitaxel (Cell Therapeutics) in which paclitaxel is bonded to a polyglutamate polymer to be more easily taken up by cancer cells; and continued early development of tumor-activated payload (TAP) technology (Novartis and ImmunoGen) in which accurate tumor targeting is achieved by the action of a monoclonal antibody specific to different tumor cells.

Until now, people with metastatic pancreatic cancer have not experienced any significant benefit from the many chemotherapeutic drugs that benefit other cancers. In 2013, nab-paclitaxel was approved for first-line treatment of metastatic pancreatic carcinoma, in combination with non-targeted gemcitabine.

Developments of different liposomal formulations of paclitaxel have been published, with some in clinical trials for ovarian, breast, lung, and pancreatic cancers as recently as 2013 (24, 25, 26).

### 3. Curcumin Liposomal and Modified Formulations for the Treatment of Cancers:

Encapsulated curcumin in a liposomal delivery system allows intravenous administration to avoid the problem of poor bioavailability after oral administration (27).

Systemic administration of a polymeric nanoparticle-encapsulated curcumin (NanoCurc) administered with free gemcitabine was reported by Bisht, S. (28).

The following review of modified curcumin formulations is excerpted from Prasad, S., Tyagi, K T, Aggarwal, B. B. Recent Developments in Delivery, Bioavailability, Absorption and metabolism of Curcumin: The Golden Pigment from Golden Spice. Cancer Res Treat. 2014; 46(1):2-18.

Curcumin-loaded human serum albumin (HSA) nanoparticles have a greater therapeutic effect than unmodified curcumin, without inducing toxicity. The intravenous administration of curcumin-loaded HSA nanoparticles also showed a greater therapeutic effect than free curcumin in tumor xenograft HCT116 models without inducing toxicity (29).

Liposomal curcumin inhibited different types of tumor growth in mouse models. It inhibited the growth of head and neck squamous cell carcinoma in a xenoengrafted mouse by the inhibition of NF-κB without affecting the expression of pAKT (30). Liposomal curcumin combined with radiation enhanced the inhibition of tumor growth in a murine lung carcinoma (LL/2) model (31). Intravenous treatment of liposomal curcumin in combination with cisplatin significantly inhibited growth of xenograft head and neck tumors in mice. The suppressive effect of curcumin was mediated through inhibition of cytoplasmic and nuclear IKKβ, resulting in inhibition of NF-κB activity (32).

Another derivative of curcumin conjugated with luteinizing hormone releasing hormone, [DLys(6)]-LHRH-curcumin, when given intravenously caused a reduction in tumor weights and volumes, while free curcumin at an equal dose failed to cause a significant reduction in tumor weights and volumes in the nude mouse pancreatic cancer model. This bio-conjugate enhanced apoptosis in tumor tissue (33).

Encapsulated curcumin with monomethoxy poly (ethylene glycol)-poly (ε-caprolactone) (MPEG-PCL) micelles also showed a stronger anticancer effect than that of free curcumin. Curcumin/MPEG-PCL micelles administered intravenously inhibited the growth of subcutaneous C-26 colon carcinoma in vivo (34).

To increase the bioavailability of curcumin, different formulations have been made. Among them, a nanoglobule-based nanoemulsion formulation has been prepared to evaluate the potential for the enhancement of solubility. In an ex-vivo study, the release of curcumin from the nanoemulsion was much higher than that of a curcumin suspension (35). Another study showed that encapsulation of curcumin into hydrogel nanoparticles yielded a homogenous curcumin dispersion in aqueous solution compared to the free form of curcumin. Also, the in-vitro release profile showed up to 95% release of curcumin from the developed nano-microparticulate systems (36).

The pharmacokinetics of nanoemulsion curcumin (NEC) containing up to 20% curcumin (w/w) showed a 10 fold increase in the area under the blood concentration-time curve (AUC) in 24 hours and more than 40-fold increase in the C (max) in NEC compared to free curcumin in mice (37).

Another curcumin-loaded apotransferrin nanoparticle (nano-curcumin), prepared by sol-oil chemistry, releases significant quantities of drug gradually over a fairly long period, 50% of curcumin still remaining at 6 hours of time. In contrast, intracellular soluble curcumin (sol-curcumin) reaches a maximum at 2 hours followed by its complete elimination by 4 hours (38).

The colloidal nanoparticles, named as ‘theracurmin’ showed an AUC after the oral administration more than 40-fold higher than that of curcumin powder in rats. In healthy human volunteers, theracurmin (30 mg), when administered orally, resulted in a 27-fold higher AUC than that of curcumin powder. The nanoparticle of curcumin prepared by Cheng et al. produced significantly higher curcumin concentrations in plasma and a six times higher AUC and mean residence time in murine brains than regular curcumin. Thus, nanocurcumin enhances bioavailability of curcumin in animals as well as in humans (39).

To improve the pharmacokinetics of curcumin with enhancing its bioavailability, another effective formulation—PLGA encapsulated curcumin—was prepared. An in-vitro study showed that PLGA-curcumin has a very rapid and more efficient cellular uptake than curcumin. Intravenous administration of either curcumin or PLGA-curcumin (2.5 mg/kg), exhibited almost a twice as high serum concentration of PLGA-curcumin than free curcumin (40).

Another formulation PLGA and PLGA-polyethylene glycol (PEG) (PLGA-PEG) blend nanoparticles containing curcumin was prepared. The PLGA and PLGA-PEG nanoparticles increased the curcumin mean half-life by approximately 4 or 6 hours, respectively, and the C (max) of curcumin increased 2.9- or 7.4-fold, respectively. Compared to the curcumin aqueous suspension, the PLGA and PLGA-PEG nanoparticles increased the curcumin bioavailability by 15.6- and 55.4-fold, respectively. Thus these formulations are potential carriers for the oral delivery of curcumin (41).

Another study showed that curcumin encapsulated in low versus high molecular weight PLGA result in relatively different oral bioavailability rates of curcumin. It has been found that the relative bioavailability of high molecular weight PLGA-conjugated curcumin is 1.67- and 40-fold higher than that of low molecular weight PLGA-conjugated curcumin or conventional curcumin, respectively (42).

After oral administration of curcumin-PLGA nanoparticles, the relative bioavailability was increased 5.6-fold and has a longer half-life compared with that of native curcumin. This improved oral bioavailability of curcumin was found to be associated with improved water solubility, higher release rate in the intestinal juice, enhanced absorption by improved permeability, inhibition of P-glycoprotein-mediated efflux, and increased residence time in the intestinal cavity (43).

It has been also observed that PLGA-curcumin effects two- and six-fold increases in the cellular uptake performed in cisplatin-resistant A2780CP ovarian and metastatic MDA-MB-231 breast cancer cells, respectively, compared to free curcumin (44).

Another formulation designed for improvement of bioavailability of curcumin is liposomal curcumin. Liposomes are considered as effective drug carriers because of their ability to solubilize hydrophobic compounds and to alter their pharmacokinetic properties. In rats, oral administration of liposome-encapsulated curcumin (LEC) showed high bioavailability of curcumin. In addition, a faster rate and better absorption of curcumin were observed as compared to the other forms. Oral LEC gave higher C (max) and shorter T (max) values, as well as a higher value for the AUC, at all time points (45). Liposome-encapsulated curcumin was evaluated in vivo and in vitro in pancreatic cancer (46).

Silica-coated flexible liposomes loaded with curcumin (CUR-SLs) and curcumin-loaded flexible liposomes (CUR-FLs) without silica-coatings have been designed. The bioavailability of CUR-SLs and CUR-FLs was 7.76- and 2.35-fold higher, respectively, than that of curcumin suspensions. Silica coating markedly improved the stability of flexible liposomes, and CUR-SLs exhibited a 3.31-fold increase in oral bioavailability compared with CUR-FLs (47).

Curcumin incorporated into N-trimethyl chitosan chloride (TMC)-coated liposomes exhibited different pharmacokinetic parameters and enhanced bioavailability, compared with curcumin encapsulated by uncoated liposomes and curcumin suspension. Uncoated curcumin liposomes and TMC-coated curcumin liposomes showed similar in-vitro release profiles (48).

In order to facilitate the intracellular delivery of curcumin, a new type of liposome-propylene glycol liposome (PGL) has been prepared. In vitro, PGL exhibited the highest uptake of curcumin compared with that of conventional liposomes and free curcumin solution (49). These studies indicate that liposome-conjugated curcumin increases the bioavailability of curcumin.

Cyclic oligosaccharides have been also used in order to improve curcumin's delivery and bioavailability via its encapsulation with Cyclodextrin (CD). It has been found that CD-encapsulated curcumin (CDC) had a greater cellular uptake and longer half-life in cancer cells compared with free curcumin indicating CDC has superior attributes compared with free curcumin for cellular uptake (50).

In addition, the improvement of curcumin permeability across animal skin tissue was observed in CD-encapsulated curcumin and was about 1.8-fold compared with free curcumin (51).

These studies suggest that CDC improves the in-vitro and in-vivo bioavailability and chemotherapeutic efficacy compared to curcumin alone.

Natural compounds have been also used to increase the bioavailability of curcumin. One of them is piperine, a major component of black pepper, known to inhibit hepatic and intestinal glucuronidation and also shown to increase the bioavailability of curcumin. This effect of piperine on the pharmacokinetics of curcumin has been shown to be much greater in humans than in rats. In humans, curcumin bioavailability was increased by 2,000% at 45 minutes after co-administering curcumin orally with piperine, whereas in rats, it has been found that concomitant administration of piperine (20 mg/kg) with curcumin (2 g/kg) increased the serum concentration of curcumin by 154% for a short period of 1-2 hours post drug administration. The study shows that in the dosages used, piperine enhances the serum concentration, extent of absorption and bioavailability of curcumin in both rats and humans with no adverse effects (52).

Most, if not all, formulated curcumin preparations have better bioavailability and biological activities than unformulated curcumin. Nanosuspension of curcumin also induces more cytotoxicity in HeLa and MCF-7 cells than curcumin (34).

Curcumin liposomes of dimyristoyl phosphatidylcholine and cholesterol inhibit the proliferation of prostate cancer cells 10 times more than unmodified curcumin (53).

Beside these, PLGA-encapsulated curcumin has shown to be more potent than curcumin in inducing apoptosis of leukemic cells and in suppressing proliferation of various tumor cell lines. It was also more active than curcumin in inhibiting TNF-induced NF-κB activation and in suppression of NF-κB-regulated proteins involved in cell proliferation, invasion, and angiogenesis (40). PLGA-nanocapsulated curcumin was found to eliminate diethylnitrosamine-induced hepatocellular carcinoma in rats (54).

Doxorubicin and curcumin in a single PLGA nanoparticle formulation has shown that curcumin facilitates the retention of doxorubicin in the nucleus for a longer period of time. It also inhibits the development of drug resistance for the enhancement of antiproliferative activity of doxorubicin in K562 cells (55).

Cyclodextrin-encapsulated curcumin (CDC) is another formulation of curcumin having anti-inflammatory and antiproliferative effects. CDC was found more active than free curcumin in inhibiting TNF-induced activation of the NF-κB and in suppressing gene products regulated by NF-κB, including those involved in cell proliferation, invasion, and angiogenesis. CDC was also more active than free curcumin in inducing the death receptors DR4 and DR5, and apoptosis (50). CD-entrapped curcuminoid also induces autophagic cell death in lung cancer cells and inhibits tumor growth in nude rats (56).

Besides these, other formulations such as dipeptide nanoparticles, and phosphatidylcholine-encapsulated curcumin, have more efficacious biological activities compared to free curcumin. A dipeptide nanoparticle of curcumin inhibits tumor growth in mice (57).

Phosphatidylcholine-encapsulated curcumin exhibits antimalarial activity (58), inhibits vaginal inflammation (59), and induces cytotoxicity of cancer cells (60).

### 4. Curcumin and Chitosan for the Treatment of Alzheimer's Disease:

An option for delivering therapeutic compounds across the BBB is the use of chitosan as a non-specific targeting molecule. This natural polysaccharide composed of randomly distributed β-(1-4)-linked D-glucosamine and N-acetyl-D-glucosamine has been patented for targeted drug delivery for treating neurodegenerative disorders. Chitosan and its biodegradable products are bioactive on nerve cells and cross the BBB, and may be developed with encapsulated curcumin to cross the BBB for treating Alzheimer's disease. Chitosan is reviewed as a suitable nanocarrier for anti-Alzheimer's drug delivery and siRNA to the brain (4).

Curcumin, a generic natural curcuminoid non-toxic antiproliferative and anti-inflammatory agent has been shown to have anti-amyloidogenic activity and induces degradation of amyloid 13 deposits and uptake by macrophages in AD. The problem with its use, however, is its poor bioavailability in oral form.

Curcumin-decorated nanoliposomes have shown high affinity for amyloid-β1-42 peptide and exhibit protective effects against Alzheimer's disease (61).

### In Summary:

As mentioned above, the present delivery of gemcitabine, paclitaxel and curcumin has been improved by various non-specific liposomal delivery systems. However, these systems are unable to directly target the APCs of the immune system to deliver the active agents intracellularly to mobilize mDCs, CTLs, circulating monocytes, macrophages, and TAMs. Successfully overcoming this shortcoming with exclusive intracellular delivery of therapeutic agents to the APCs permits the enhancement of a cascade of immunotherapeutic events to disease onset and progression, and also mobilizes the APCs to act as messengers that transport the therapeutic agents to disease sites in the body and to the brain.

### SUMMARY OF THE INVENTION

Targeted delivery is performed to accomplish:

1. Treatment of Malignant Diseases:
Exclusively target cellular membrane-expressed C-type lectin receptors (CLRs) in vivo to enable intracellular entry by clathrin-mediated endocytosis of three agents in a combination approach to treat cancers: CLR-TargoSphere-encapsulated antiproliferative and anti-inflammatory agents (gemcitabine, paclitaxel, and/or curcumin) preferentially or even exclusively into myeloid antigen-presenting cells (APCs) by extracellular fluids, i.e., lymph, tissue fluid, and/or blood with distribution to the secondary lymphatic organs and tumor sites.

2. Treatment of Neurodegenerative and Demyelinating Diseases of the Central and Peripheral Nervous System:
Exclusively target the CLR-TargoSphere-encapsulated antiproliferative and immune-enhancing agent paclitaxel and the CLR-TargoSphere-encapsulated anti-inflammatory agent curcumin to cross the BBB to treat neurodegenerative diseases such as Alzheimer's disease in a dual treatment strategy.

The term “CLR-TargoSphere” refers to a lipid-based nanocarrier furnished with surface-embedded targeting ligands consisting of a CLR-targeted carbohydrate linked to cholesterol. Said targeted lipid-based nanocarrier affords an internal aqueous space into which hydrophilic actives can be encapsulated and dissolved. Hydrophobic or amphiphilic actives can be embedded in whole or in part within the nanocarrier's outer surface double membrane.

The preparation of the CLR-TargoSphere is described in detail in US 2007/0292494 A1 and within the knowledge of the person skilled in the art. For example, nanocarriers are formulated according to a basic protocol published before (Gieseler R K et al. Mar. 21, 2005; WO 2005/092288 A1). However, protocols may be modified in that the surface densities of targeting anchors can be varied between 5% and 10% surface density of the Fucose-derivative ligand for addressing cells via the CLRs expressed on their surface. The aforementioned patent applications and references are incorporated herein by reference.

Technical problems addressed by this invention:
(1) Develop three individually encapsulated antiproliferative, immune-enhancing, and anti-inflammatory products in the CLR-TargoSphere:
   a) Paclitaxel, an antiproliferative and immune-enhancing hydrophobic agent embedded into the lipid bilayer of a CLR-TargoSphere;
   b) Gemcitabine, a hydrophilic antiproliferative agent encapsulated into the aqueous core of a CLR-TargoSphere;
   c) Curcumin, an antineoplastic, immune-enhancing, and anti-inflammatory hydrophobic agent embedded into the lipid bilayer of a CLR-TargoSphere.
(2) Exclusively target the CLRs in vivo to enable entry by clathrin-mediated endocytosis of CLR-TargoSphere-encapsulated contents (paclitaxel and curcumin) into APCs by extracellular fluids, i.e., lymph, tissue fluid, and/or blood with distribution to tumor sites and across the BBB into the CNS' perivascular spaces, glial cells and astrocytes.
(3) The three encapsulated agents (paclitaxel, gemcitabine, and curcumin) are delivered by extracellular fluids to the mDCs, circulating monocytes, macrophages, TAMs, and importantly, to the secondary lymphatic organs, where lymphocytes are activated and instructed by the mDCs and monocytes encapsulating curcumin to enhance induction of antigen-specific cancer cell programmed death (PD-1)-positive CTLs. The active agents are also shuttled to tumor sites by means of circulating mDCs and monocytes, where they generate multiple host cellular and cytokine therapeutic responses, resulting in enhancement of tumoricidal macrophages (M1), apoptosis of cancer cells, increased tumor sensitivity to gemcitabine, decreased development of tumor resistance, inhibition of angiogenesis, inhibition of tumor migration, inhibition of genetic transformation of cancer cells, reduction in tumor growth, and inhibition of metastases.
(4) CLR-targeted delivery of two of the encapsulated agents (paclitaxel and curcumin) to circulating monocytes and macrophages, which are known to cross inflamed endothelial walls of the BBB, a hallmark of neuroinflammatory disease; to be delivered to perivascular spaces, glial cells and astrocytes of the CNS to inhibit neuroinflammatory amyloid 13 aggregation and plaque formation, and abnormal tau hyperphosphorylation, both elements associated with impaired synaptic transmission, neurofibrillary entanglement, and neurodegeneration in Alzheimer's disease (curcumin has potent anti-amyloidogenic effects for Alzheimer's β-amyloid fibrils in vitro: Kenjiro Ono, et. al. J of Neuroscience Research, DOI.1002/jnr.20025; 75:742-750, 15 March 2004).

### Advantages Presented by this Invention

(i) Highly specific targeted intracellular delivery of tailored combinations of antiproliferative and anti-inflammatory agents to communicating cells of the immune system that migrate to cancer sites and/or neurodegenerative disease sites, thereby shuttling the active agents to these same disease sites for cellular and immunotherapeutic interactions;
(ii) Targeting all CLRs, and hence, APCs as the communicating cells of the immune system, and thereby addressing all CLR-mediated ports of cell entry;
(iii) Addressing all myeloid APC species (i.e., mDCs and macrophages of all developmental tissue stages, as well as peripheral blood monocytes;
(iv) Generate a broad spectrum of immune-defensive responses to malignant and neurodegenerative diseases with the production of therapeutic cytokines, lymphokines, growth factors, enzymes, transcription factors, inflammatory mediators, and protein kinases; and
(v) Limit systemic circulation of therapeutic agents with decreased dosing requirements for achieving therapeutic goals, and thereby offering decreased systemic toxicities.

### BRIEF DESCRIPTION OF THE FIGURES

FIG. 1

Potential routes of (functionalized) uptake of lipid-based nanocarriers by APCs

Carriers are efficiently internalized by macrophages (Mt), monocytes (M), and dendritic cells (DC). Depending on the presence of glycan targeting-ligands on their surface, the internalization pathways involved may differ. Non-targeted nanocarriers could be internalized through macro-pinocytosis (1) or direct membrane fusion (2), whereas glycosylated carriers may be taken up additionally and/or preferentially through CLR-mediated endocytosis (3), entering endo-lysosomal pathways to the endoplasmic reticulum (ER). Depending on the uptake pathway, subsequent intracellular processing may differ. (Source: Frenz T, Grabski E, Durán V, Hozsa C, Stȩpczyńska A, Furch M, Gieseler R K, Kalinke U. Antigen presenting cell-selective drug delivery by glycan-decorated nanocarriers. Eur J Pharm Biopharm 2015. Pii: S0939-6411(15)00090-9. Doi: 10.1016/j.ejpb.2015.02.008).

FIG. 2a, and FIG. 2b (A, B, C, D, E, F, G)

Lewis (LEW) rat brain transmission electron microscopy showing uptake into astrocytes of an active pharmaceutical agent across the blood-brain barrier upon subcutaneous delivery of API-loaded CLR-TargoSpheres.

FIG. 2a: TargoSphere-dependent cell targeting in the brain. Left: TS delivered fluorescent RBT-05 to discrete cell groups. In the image on the right-hand side, RBT-05 fluorescence and haematoxylin-stained cell nuclei are superimposed. RBT-05 was never seen distributed evenly throughout the brain, but was always confined to isolated groups of cells. At this magnification, 1 cm corresponds to approx. 100 μm.

FIG. 2b (A, B, C, D, E, F, G): Transmission electron microscopy (TEM): Demonstration of crossing of the blood-brain barrier (BBB) by the TargoSphere (TS).

FIG. 3

Gemcitabine

FIG. 4

Paclitaxel

FIG. 5

Curcumin

FIG. 6

Paclitaxel mannose analogue (AB-1)

FIG. 7

Synthesis of AB-1

a) 2,2-dimethoxypropane, p-TsOH, DMF, r.t., 88% yield; b) AcOH, water, r.t., 85% yield; c) CBr₄,Ph₃P, CH₂Cl₂, r.t., 80% yield; d) NaH, DMF, KI (cat.), 90° C., 57% yield; e) 20% HCl, 60-65° C., 100% yield; f) TBDMSCI, imidazole, DMF, 100-110° C., 30-50% yield; g) 10% Pd/C, H₂, r.t., 93% yield; h) bis(4-nitrophenyl)carbonate, EtN(iPr)₂, CH₂Cl₂, 80% yield; i) Paclitaxel, DMAP, CH₂Cl₂, 78% yield; j) 1M TBAF, AcOH, THF, 40-50% yield.

### DETAILED DESCRIPTION OF THE INVENTION

This provisional patent application will be filed as an extension to patent EP 05 725 950.0 and U.S. Ser. No. 10/593,355 which are incorporated herein per reference.

Generally, the present invention relates to targeted nanocarriers—also termed nanomedicines—and methods of preferentially, or actively, targeting and delivering gemcitabine, paclitaxel and/or curcumin (i.e. any compound alone but also any possible combination thereof) to a range of mammalian cell species. Cell-specific targeting is achieved by using nanocarriers featuring a Fucose-derivative targeting anchor. Preferably, the anchor is Fucose-4-Chol. Such targeting anchor may or may not include a polymeric spacer like polyethylene glycol. The nanomedicines shall allow to therapeutically address a range of mammalian disease entities via various application routes. These indications include malignant diseases and neurodegenerative or demyelinating diseases.

The invention involves the manufacture of three individual products which will require:
(1) Encapsulating gemcitabine into the aqueous compartment of the CLR-TargoSphere;
(2) Embedding paclitaxel into the lipid layer of a CLR-TargoSphere;
(3) Embedding curcumin into the lipid layer of a CLR-TargoSphere.

The CLR-TargoSphere-embedded or -encapsulated agents are administered in separate combinations, in parallel, or in alternating regimens to target APCs. In a preferred embodiment, the mode of delivery of the nanocarrier is via an intravenous, a subcutaneous, an intratumoral, an intrametastatic, an intradermal, an intraperitoneal, a parenteral, a transdermal, or an intrapulmonary route, a route by infusion via the hepatic artery, an intrathyroidal route, an intranasal route, an intrathecal route, or a topical route. In a particular preferred embodiment the mode of administration is parenterally.

APCs are responsible for host defense against immunorelevant diseases. They communicate directly with tumors and neurodegenerative tissues. They produce a broad spectrum of therapeutic cytokines, lymphokines, growth factors, enzymes, transcription factors, inflammatory mediators, and protein kinases in response to neurodegenerative or malignant diseases. Delivering the targeted antiproliferative and/or anti-inflammatory agents directly to the APCs will enable effective immunotherapeutic treatment of malignancies and neurodegenerative diseases, as well as targeted delivery of antiproliferative and anti-inflammatory agents to diseased cells and tissues.

The malignant diseases are, e.g., metastatic pancreatic adenocarcinoma, triple-negative breast cancer, small cell lung carcinoma, malignant melanoma, head and neck squamous cell carcinoma, renal cell carcinoma, prostate cancer, bladder cancer, small and large bowel carcinoma, thyroid carcinoma, non-Hodgkin's lymphoma, the leukemias, cervical carcinoma, ovarian carcinoma, Kaposi's sarcoma, osteosarcoma, basal cell carcinoma, and squamous cell carcinoma.

The neurodegenerative diseases include, e.g., Alzheimer's disease, Parkinson disease, spinal cord trauma, stroke, Huntington's disease, amyotrophic lateral sclerosis, and multiple sclerosis, including the class of demyelinating diseases.

### General Mode of Action

Three CLR-TargoSphere formulations identical in composition yet differing in their payloads (i.e., gemcitabine, paclitaxel and/or curcumin, respectively) are manufactured. These formulations shall be administered either together or sequentially. If to be given sequentially—which is the most likely scenario—the specific sequence evoking the desired therapeutic effect will have to be determined experimentally as no information on the best result in a given indication is currently known. In addition, the concentrations of the different TargoSphere/API formulations to be administered for optimizing this best therapeutic result will also have to be determined experimentally. Optimized results are defined by the treatment objectives specified hereinafter.

The present invention is originative due to the fact that no information on how to achieve an optimal outcome for a given indication with the three aforementioned components is known at this time. Hence, no person skilled in the art would currently be able to deduce the presumptive outcome of such treatment from earlier results on the administration of either (i) the freely soluble active agents or (ii) the nanocarrier-encapsulated agents. In fact, no combination of these three APIs has thus far been tested and none of the nanocarriers already employed with either of these APIs has the same targeting characteristics as does the CLR-TargoSphere. While the potential scope of aspects contributing to the final outcome of these novel combinatorial treatment variants are indeed illustrated by earlier results, the concrete aspects triggered by such treatments, their magnitude, as well as any complementary synergistic effects cannot be anticipated.

A variation of the CLR-TargoSphere, a non-encapsulated mannosylated analogue of paclitaxel (AB-1), was developed and delivered intravenously to male athymic NCr-nu/nu mice that had been implanted with U251 human glioblastoma cells intracerebrally. The paclitaxel analogue was developed with mannose attached to its surface in order to efficiently attach to mannose receptors on migrating monocytes and be delivered to the implanted glioblastoma brain tumors by means of circulating monocytes, which are known to cross leaky blood vessel walls in the tumor microenvironment and thereby the BBB to deliver the active antineoplastic agent directly to the tumor environment (see FIG. 6).

Objectives for the Treatment of Malignancies and/or Neurodegenerative Diseases:
(1) Targeting the APCs of the immune system intracellularly to achieve effective and highly specific shuttling of the antiproliferative and anti-inflammatory agents to the site of disease and avoid systemic delivery and associated systemic toxicities, with reduced dosages and improved efficacy;
(2) Intracellular delivery of gemcitabine, paclitaxel, and curcumin to C-type lectin receptor-positive APCs in order to enhance potent cellular and immune-therapeutic host responses for a variety of tumors for which gemcitabine and paclitaxel have already received regulatory approval, including, metastatic pancreatic adenocarcinoma, triple negative breast cancer, small cell lung carcinoma, malignant melanoma, head and neck squamous cell carcinoma, renal cell carcinoma, prostate cancer, bladder cancer, small and large bowel carcinoma, thyroid carcinoma, non-Hodgkin's lymphoma, the leukemias, cervical carcinoma, ovarian carcinoma, Kaposi's sarcoma, osteosarcoma, basal cell carcinoma, and squamous cell carcinoma;
(3) To inhibit DNA synthesis and development of genetic resistance by cancer cells with targeted gemcitabine, paclitaxel, and curcumin;
(4) For paclitaxel to induce apoptosis by its action as a microtubule stabilizer and to enhance the generation of tumoricidal (M1) TAMs;
(5) For curcumin to promote induction of tumor antigen-specific PD-1-positive CTLs to attack tumor cells, and to induce apoptosis by mitochondrial hyperpolarization at the tumor site, inhibit neovascularization, and increase tumor sensitivity to gemcitabine, thereby inhibiting the development of tumor resistance;
(6) To exclusively target the combination paclitaxel and curcumin to mDCs, migrating monocytes and tissue macrophages to cross the inflamed BBB of the CNS for the treatment of the neurodegenerative and neuroinflammatory diseases, such as Alzheimer's disease. The mDCs, monocytes, macrophages, and TAMs migrate to the inflamed endothelial walls of the CNS and shuttle the active agents across the inflamed BBB, thereby reaching the perivascular spaces, glial cells and astrocytes. Curcumin is delivered to the CNS to inhibit amyloid 13 formation, aggregation, and deposition; and paclitaxel is delivered to inhibit production of abnormal hyperphosphorylated tau protein, and prevent tau-induced synaptic transmission pathology;
(7) To reduce microglial proliferation, differentiation, and amyloid 13 deposition in Alzheimer's disease with the combination of paclitaxel and curcumin delivered by mDCs, monocytes, and macrophages across the BBB;
(8) To deliver curcumin and paclitaxel to the CNS and/or spinal cord, across the BBB as a potent anti-inflammatory in neurodegenerative diseases, spinal cord trauma, stroke, and neuroinflammatory diseases of the CNS including Parkinson's disease and multiple sclerosis;
(9) To induce apoptosis of tumor cells by the complementary actions of gemcitabine and paclitaxel in inhibiting DNA replication and curcumin by the mitochondrial pathway;
(10) To inhibit early chemotherapy resistance to gemcitabine and/or paclitaxel with the combination of paclitaxel and gemcitabine;
(11) To enhance promotion of tumoricidal M1 TAMs with paclitaxel targeted to circulating monocytes and delivered to the tumor stroma;
(12) To inhibit angiogenesis with the combination of targeted paclitaxel and curcumin to tumor sites;
(13) To induce apoptosis with curcumin-enhanced induction of tumor antigen-specific PD-1-positive CTLs;
(14) To induce apoptosis by mitochondrial hyperpolarization with curcumin;
(15) To enhance effective drug concentrations and cytotoxicity at the tumor sites by the targeted delivery of gemcitabine, paclitaxel, and curcumin to APCs;
(16) To reduce systemic delivery and adverse effects to healthy bystander cells by targeted delivery and the avoidance of systemic distribution of the antiproliferative drugs;
(17) To prevent development of resistance to gemcitabine with targeted delivery of paclitaxel and curcumin, a combination of antiproliferative agents, each having different yet synergistic mechanisms of action;
(18) To safely attempt to increase duration of treatment, when indicated, with decreased dosing while also achieving an improved anti-tumor response;
(19) To increase progression-free survival and overall survival;
(20) To decrease systemic toxicities, increased drug concentrations in targeted tissues, decreased metabolic elimination, increased half-life, improved patient compliance, and improved clinical outcomes.
(21) Because curcumin is poorly bioavailable in oral form, CLR-TargoSphere delivery will allow for the successful bioavailable delivery of curcumin to enable the following therapeutic antiproliferative and anti-inflammatory actions:
It enhances induction of tumor antigen-specific, PD-1-positive CTLs (62, 63);
It arrests cancer cells in various phases of the cell cycle, and induces apoptosis primarily through a mitochondrial pathway involving caspase-8-dependent BID cleavage (64);
It has been shown to inhibit constitutive NF-κB activation, induce G1/S arrest, suppress proliferation, and induce apoptosis in mantle cell lymphoma (65);
It induces apoptosis in human melanoma cells through a Fas Receptor/Caspase-8 pathway independent of p53 (66);
It suppresses the proliferation of human vascular endothelial cells in vitro and inhibits the Fibroblast Growth Factor-2-induced angiogenic response in-vivo (67);
It induces apoptosis in the human acute myelogenous leukemia cell line HL-60, believed to occur through the mitochondrial pathway involving caspase-8, BID cleavage, cytochrome c release, and caspase-3 activation (68);
It downregulates action of NF-κB and the antiapoptotic genes regulated by NF-κB, a critical role in inhibiting cancer cell survival and proliferation in pancreatic cancer (69, 70);
It suppresses expression of NF-κB, Bcl-2 and Bcl-XL in multiple myeloma cell lines (71);
A liposome-encased formulation of curcumin was studied in pancreatic cancer cell lines in vitro and in vivo, by intravenous infusion, in athymic mice at the M. D. Anderson Cancer Center in Houston, Tex. Liposomal curcumin was shown to down-regulate the NF-κB machinery, suppress tumor growth, and induce apoptosis in vitro, and demonstrated a reduction in tumor burden and angiogenesis in vivo (72);
It potentiates the antitumor activity of gemcitabine in an in-vivo pancreatic cancer model through suppression of proliferation, angiogenesis, and inhibition of NF-κB-regulated gene products (73);
It induces gemcitabine sensitivity in pancreatic cancer cells by modulating miR-200 and miR-21 expression (74);
It inhibits tumor growth and angiogenesis in an orthotopic mouse model of human pancreatic cancer (75);
It induces apoptosis in squamous cell carcinoma head and neck cell lines with suppression of NF-κB, cell proliferative genes including Bcl-2, cyclin D1, IL-6, COX-2, and MMP-9 (76).
Curcumin has potent anti-amyloidogenic effects for Alzheimer's β-amyloid fibrils in vitro (77).
The major mechanism by which curcumin kills malignant cells is via its pro-apoptotic action different from, but complementary to the mechanisms of paclitaxel or gemcitabine. This action is mainly through a mitochondrial pathway involving caspase-8, BID cleavage, cytochrome c release, and caspase-3 activation. Curcumin is also critically important in enhancing production of tumor antigen-specific PD-1-positive CTLs, thereby enhancing cancer cell apoptosis. Targeted curcumin to astrocytes and glial cells in the CNS bypasses the poorly bioavailable form of oral curcumin to achieve anti-amyloidogenic activity.

### EXAMPLES

### Example 1: Preparation of CLR-TargoSpheres

### Reagents:

DOPC (1,2-Dioleoyl-sn-glycero-3-phosphocholine), DMPC (1,2-Dimyristoyl-sn-glycero-3-phosphocholine), DMPG (1,2-Dimyristoyl-sn-glycero-3-phospho-rac-glycerol) and unsaturated phospholipids were purchased from Lipoid GmbH (Ludwigshafen, Germany). Cholesterol and curcumin, were obtained from Sigma-Aldrich Chemie GmbH (Munich, Germany). Paclitaxel and gemcitabine hydrochloride were purchased from LC Laboratories (Woburn, USA). The CLR-targeting lipid was synthesized by Merck & Cie (Schaffhausen, Switzerland). Buffer salts, ethanol, methanol and chloroform were purchased from Carl Roth GmbH & Co. KG (Karlsruhe, Germany). PBS buffer tablets (pH 7.4) were obtained from VWR (Darmstadt, Germany). Polysorbate 80 and PEG 400 were bought from Caesar & Loretz GmbH (Hilden, Germany). Texas Red DHPE (Texas Red 1,2-dihexadecanoyl-sn-glycero-3-phosphoethanolamine, triethylammonium salt) was provided by Life Technologies GmbH (Darmstadt, Germany). Unless specified otherwise, all chemicals were of analytical or higher grade. Generally, ultrapure water (ρ=18.2 MΩ·cm), produced with a Milli-Q water filtration station (Merck KGaA, Darmstadt, Germany) was used for all preparations.

### TargoSphere Preparation and Drug Encapsulation

CLR-TargoSpheres Loaded with Gemcitabine:

Gemcitabine cannot be stably entrapped into conventional liposomes. Due to its physicochemical nature it will rapidly diffuse through liposomal bilayers [79]. With dual asymmetric centrifugation, however, approx. 33-40% of a given gemcitabine amount can be entrapped in a vesicular phospholipid gel (VPG) [79, 82]. This gel contains highly concentrated gemcitabine-loaded liposomes and can be diluted immediately before use to obtain a solution of individual liposomes.

CLR-TS:Gemcitabine were prepared by using a dual asymmetric centrifugation method (DAC [82]) adapted from [79]. Briefly, the lipid components DOPC, cholesterol, and CLR-targeting lipid were dissolved in the appropriate organic solvents. The x(CLR-targeting lipid) value typically varied between 0.02 and 0.16. The fluorescent dye, Texas Red DHPE, as required for binding studies (x(TR DHPE)=0.001), was dissolved in methanol. Solutions were combined in a round-bottomed flask. Organic solvents were then removed using a rotary evaporator to obtain a thin lipid film. Residual solvent was removed using a vacuum pump overnight.

The lipid film was detached from the wall of the round-bottomed flask and was transferred to a 2 mL centrifuge tube. About 200 mg ceramic beads (d=1.2-1.4 mm; Sigmund Lindner GmbH, Warmensteinach, Germany) were added. The lipid mixture was then hydrated for 10 min with phosphorous buffer (pH=7.4), containing gemcitabine hydrochloride. Homogenization was performed in a dual asymmetric centrifuge (DAC 150 FVZ, Hauschild & Co KG, Hamm, Germany) in multiples of 5-min runs at maximum speed (i.e. 3,540 rpm). In case that unsaturated lipids were used, the hydration and DAC were performed at 70° C. The resulting gel-like liposomal preparation was diluted with buffer and vigorously vortexed immediately before use.

In an alternative method, an “empty” VPG was prepared without the addition of gemcitabine during DAC. Then, a gemcitabine solution was added and the gel was mixed thoroughly. To increase the gemcitabine diffusion rate into the liposomes, the preparation was incubated for 4 h at 60° C.

If necessary, non-encapsulated drug was removed from the diluted liposomal suspension by gel filtration through a Sepharose CL-4B column (GE Healthcare Europe GmbH, Freiburg).

In general, the liposomes varied between 150 nm and 170 nm in diameter (PCS).

CLR-TargoSpheres Loaded with Curcumin:

The preparation of curcumin-loaded liposomes followed the general procedures outlined in [83] and for the production of gemcitabine-loaded liposomes, except for the following modifications: First, curcumin was dissolved in a mixture of chloroform and methanol (3:1) and added to the lipid mixture (normally, DMPC:DMPG 9:1, supplemented with 8% CLR-targeting ligand) before film preparation. The lipid mixture typically contained 10% curcumin. Second, the lipid film was hydrated with phosphorous buffer (pH=7.4). The preparation was kept on ice and protected from light, whenever feasible.

CLR-TargoSpheres Loaded with Paclitaxel:

Paclitaxel-loaded CLR-TS were prepared according to a conventional thin-film hydration and extrusion method, which was modified to take the extremely low solubility of the drug in aqueous media into account [81, 85]. In short, a thin lipid film was prepared according to the methods outlined above and [85]. The lipids (typically, 90% S PC, 2 cholesterol, and 8% CLR-targeting ligand) and paclitaxel were dissolved in chloroform. The x(PLX) value varied between 1 and about 5%. To the dried film, 1 mL PBS (adjusted to pH=4.0), supplemented with 3% polysorbate 80 and 5% PEG 400, was added for hydration. The lipid solution was briefly sonicated and then extruded 31-times over a 80 nm polycarbonate membrane. All solutions with x(PLX) 2% were clear and slightly opaque. Preparations with a higher PLX content contained fine PLX-crystal needles and were not used for further experiments. Typical properties for a x(PLX)=2% preparation were:

Z-Ave=97.8 nm, d(main component)=106 nm, PDI=0.069, c(lipids)=69 mM, γ(lipids)=53 g/L; c(PLX)=1.38 mM, γ(PLX)=1.18 g/L. A drug content of 2% translates to a maximum dose of 235 μg paclitaxel at this liposome concentration if the injection volume is 200 μL.

### Example 2: CLR-TargoSpheres Cross the Blood-Brain Barrier

The inventors employed CLR-TargoSpheres (TS) encapsulating an exploratory active agent termed RBT-05 (further dubbed TS/RBT-05), which can be visualized via an RBT-05-specific antibody. In Lewis (LEW) rats, the overall biodistribution of TS, freely soluble RBT-05, and TS/RBT-05 was determined (FIG. 2a). As evidenced below, this study demonstrated that TS/RBT-05 administered SC—but not non-encapsulated RBT-05—crosses the BBB whereupon targeting and delivering RBT-05 into discrete cells in the CNS. Besides the BBB-lining endothelial cells, the ultimate CNS-resident target cells are astrocytes and may also include activated microglia. Both cell types play important roles in the initiation and/or propagation of neurodegenerative and neuroinflammatory diseases.

Animals and Treatment Conditions: In a seven-day dose-escalation study, LEW rats were treated daily with (i) TS-encapsulated RBT-05 (verum); (ii) TS-encapsulated Dextran 10,000 (vehicle control); or (iii) non-encapsulated RBT-05 (non-targeted delivery control). Subcutaneous injections were placed under the neck skin without anesthesia. Non-encapsulated RBT-05 was applied daily at concentrations 17.8-fold of those administered in TS-encapsulated form. Verum and mock-loaded TS were applied at identical concentrations.

### Box 1: Experimental Frame Conditions.

Study Type: 7-day linear dose escalation study
Species/Strain: LEW rats (RT1Al)
Gender: Male
Total number of animals: 24
Treatment groups/sample: Three groups at n = 8, each
Body weight (BW) at onset: ~350 g
Treatment route: Subcutaneously (SC)
Injection volume: 0.5 mL per SC injection per day

### Organ Preparation and Cryosectioning:

On day 7, the rats were sacrificed by applying isoflurane (2-chloro-2-(difluoromethoxy)-1, 1, 1-trifluoro-ethane) plus an overdose of Ketamine (100 mg/kg BW) and Xylazine (20 mg/kg BW). Subsequently, organs were prepared and asservated appropriately, and were kept at −80° C. until being subjected to cryostat sectioning. Cryosections were prepared on a motorized Leica CM-3050S cryostat (Wetzlar, Germany). Depending on the type of tissue, sections were cut at 6 μm to 10 μm, with brain sections at 10 μm.

### Immunohistochemistry and LASER Scanning Microscopy:

The complete safety/toxicity and biodistribution study comprised immunohistochemistry for numerous cell-determining markers. In the present context however, only staining for the active agent, RBT-05, was of relevance. Briefly, after cell permeabilization intracellular TS-delivered RBT-05 was visualized by applying primary polyclonal rabbit anti-RBT-05, followed by secondary goat anti-rabbit IgG and IgM×FITC, and nuclear counterstaining with hematoxylin. Stained sections were evaluated under a LASER scanning microscope (LSM 510; Zeiss, Oberkochen, Germany).

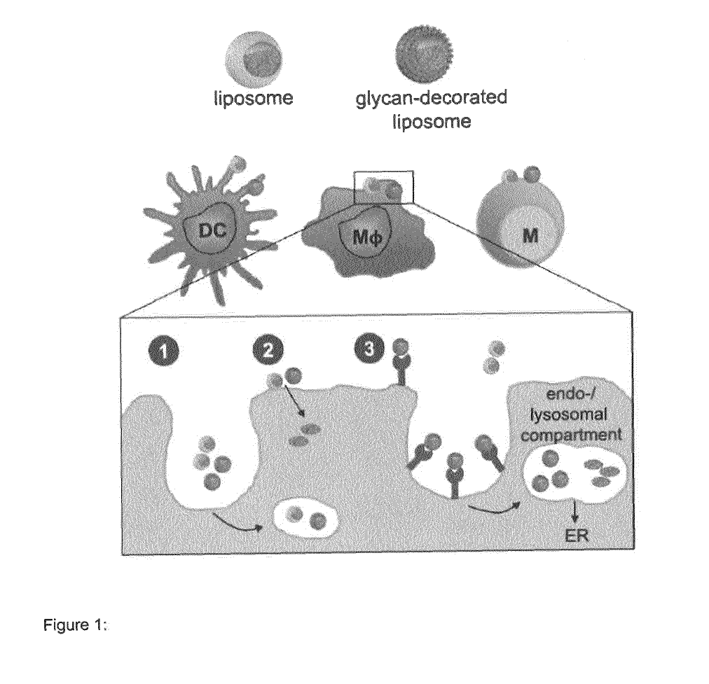

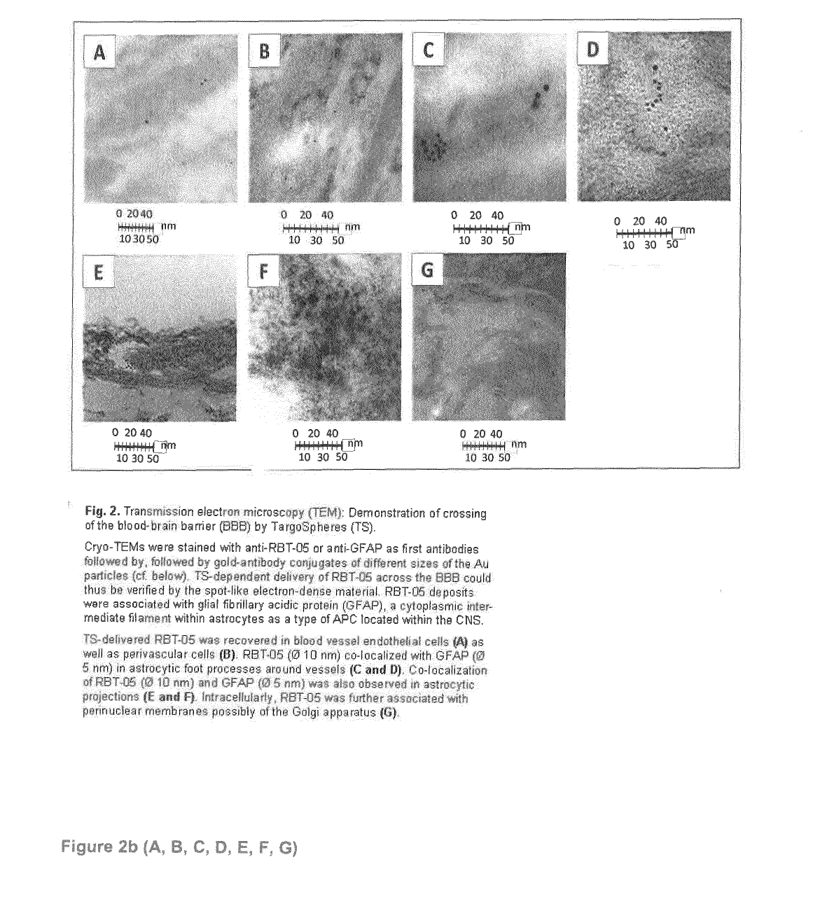

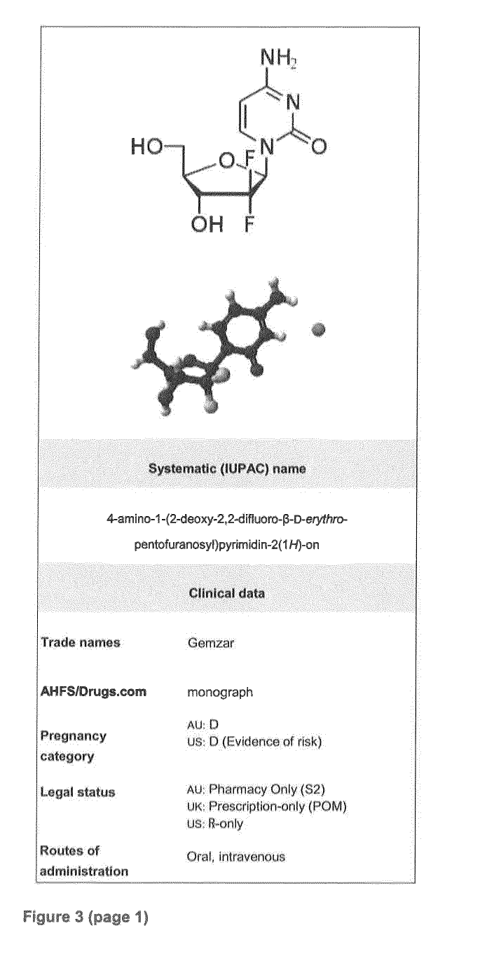

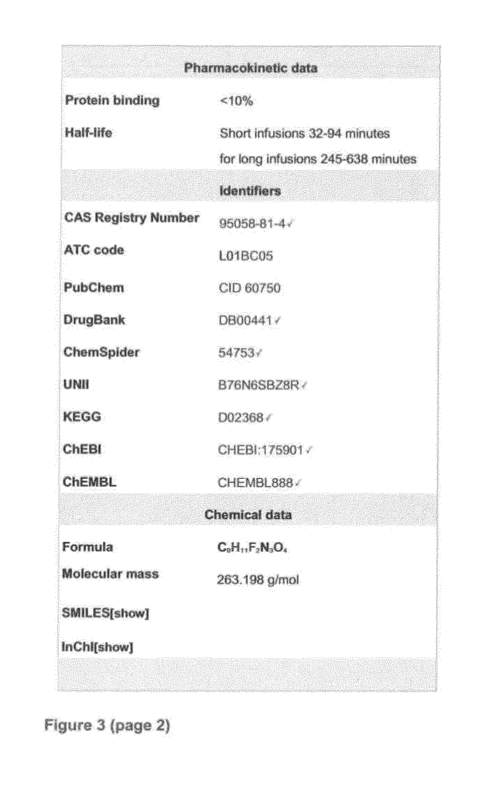

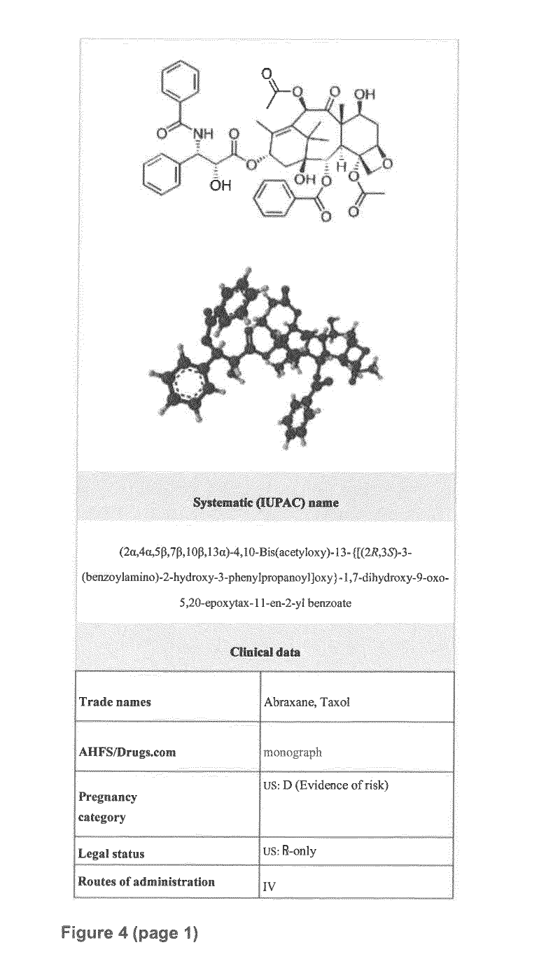

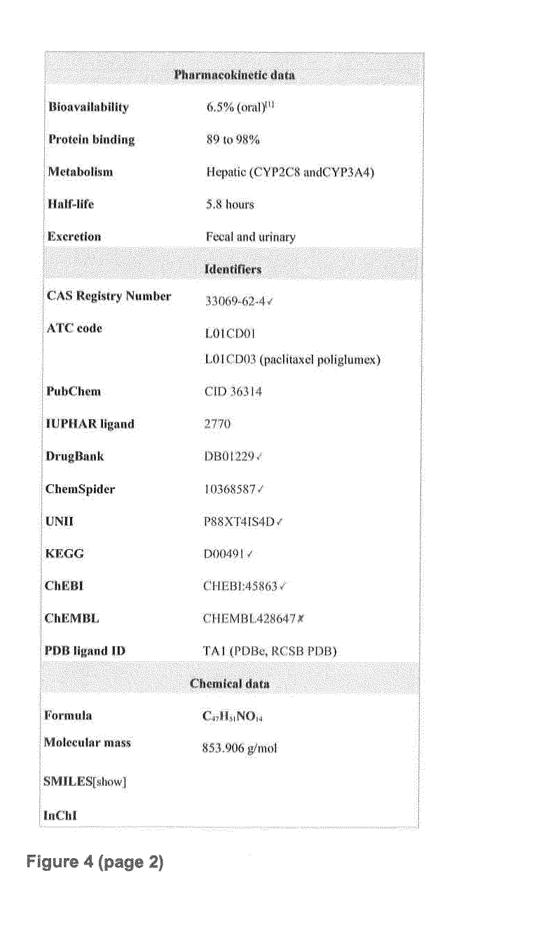

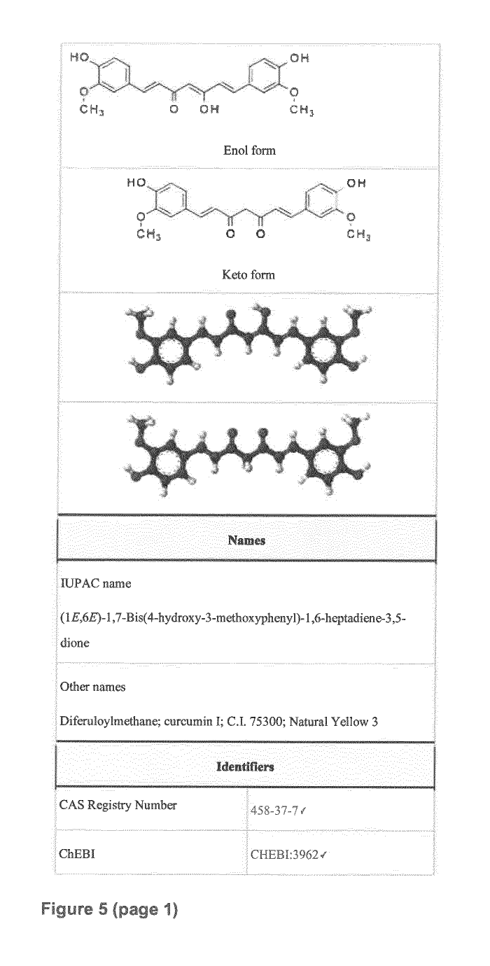

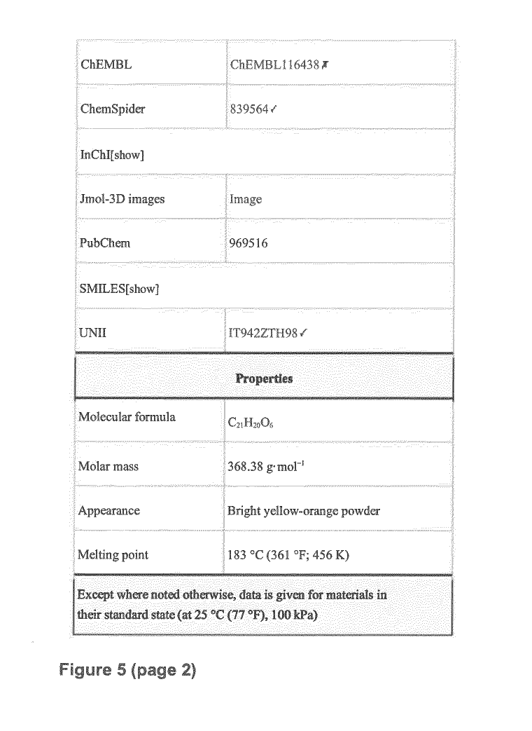

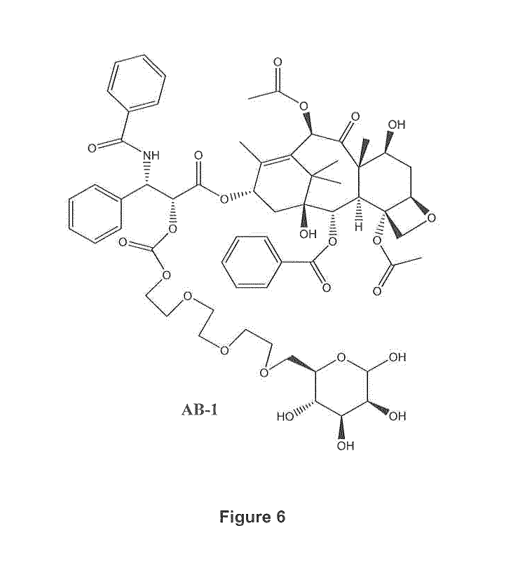

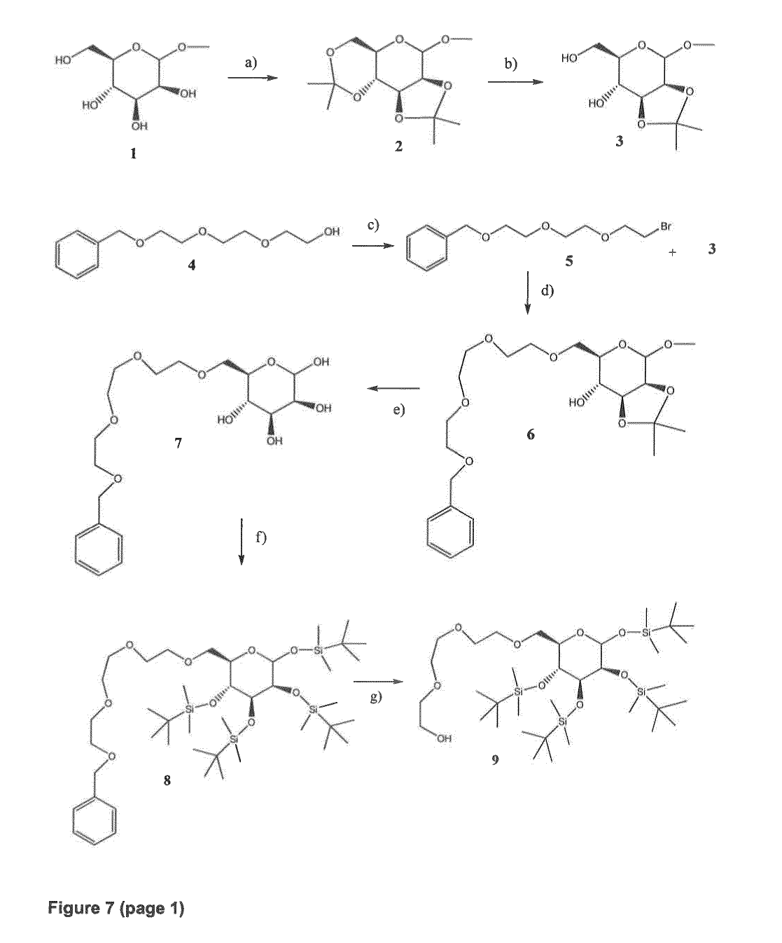

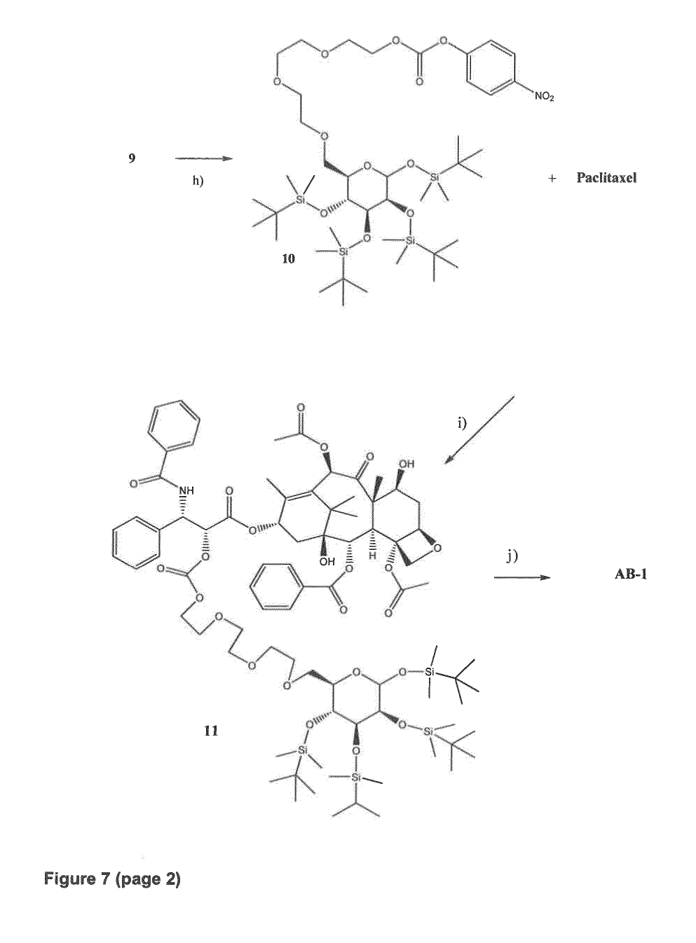

Areas comprising groups of cells in the rat brain clearly stained positive for intracellular RBT-05 (FIG. 2-1). It was thus apparent that a yet-to-be-determined amount of TargoSpheres crosses the blood-brain barrier and addresses certain brain-resident cells.

### Transmission Electron Microscopy:

Following first indications of TargoSphere-dependent crossing of the BBB by LASER scanning microscopy, we decided to further verify this finding by electron-microscopic studies. Asservated tissue blocks from the same treated animals were processed for transmission electron microscopy (TEM). Of seven frozen CNS tissue blocks from animals treated with TS/RBT-05, five intact specimens were processed, while two sections were not investigated due to fragmentation or damage. Briefly, blocks of 0.5 mm were fixed with glutaraldehyde/paraformaldehyde, dehydrated, and embedded in LR white. Fifty-nm sections were then placed upside down on nickel grids and serially incubated with different buffers. The sections received primary antibodies, followed by secondary gold (Au)-antibody conjugates, with ØAu=10 nm for secondary anti-RBT-05, and Ø Au=5 nm for secondary anti-glial fibrillary acidic protein (GFAP). Sections were washed and buffered, incubated with uranyl acetate, and air-dried.

In conventional TEM images (not shown here), antibody-reactive astrocyte processes were observed, but labeled microglia were not identified. In four out of the five specimens investigated, both RBT-05 and astrocyte-specific GFAP were detected. Specifically, anti-RBT-05 was labeled with 10-nm gold particles, while anti-GFAP was marked with 5-nm gold particles.

The TS payload protein, RBT-05, was found in blood vessel endothelial cells (FIG. 2b-A) as well as perivascular cells (FIG. 2b-B). Throughout the CNS, RBT-05 and GFAP co-localized in astrocytic foot processes and around vessels (FIG. 2b-C and FIG. 2b-D). Co-localization of RBT-05 and GFAP was furthermore observed in astrocytic processes provided that filaments were visible (FIG. 2b-E and FIG. 2b-F). Finally, RBT-05 was found associated with perinuclear membranes, possibly of the Golgi apparatus (FIG. 2b-G).

Since RBT-05 was not detectable in other CNS-resident cell populations, these cells were obviously not targeted by the TS. Besides endothelial cells (likely via their binding to selectins during BBB crossing), TS therefore specifically recognized astrocytes and may also target microglia.

Overall, the purpose of these investigations was to clarify and determine whether CLR-TargoSpheres have the capacity to cross the BBB and to target certain cells in the CNS. Our results reveal that TS indeed do cross the BBB. Importantly, the TS did not deliver its payload RBT-05 at random, but addressed certain cells as determined from the delivery pattern of RBT-05 in the CNS. Specifically, transmission electron microscopy demonstrated that the TS-delivered RBT-05 co-localized with glial fibrillary acidic protein, which is a marker for astrocyte processes.

Astrocytes have antigen-presenting properties and thus play a role in immunologically mediated inflammatory diseases in the CNS. The fact that TS-delivered payload was also observed in blood vessel-lining endothelial cells as well as perivascular cells further supports the contention of (active) BBB crossing by CLR-TargoSpheres.

### Example 3: Preparation of Paclitaxel-Triethoxy Mannose Analog (AB-1)

Reference is made to FIG. 7.

Overview:

Alcohol intermediate 3 was prepared by the literature procedure (in comparable yields) in two steps from 1-methyl-D-Mannopyranoside (TCI America) [80]. Alcohol 3 was alkylated with bromide 5 using sodium hydride in acceptable yield (57% yield). Bromide 5 was readily prepared from the commercially available alcohol (TCI America) in the presence of carbon tetrabromide and triphenylphosphine. The protective groups on ether 6 were replaced with tert-butyldimethylsilyl groups that would be easier to remove in the final step to prepare the silylated ether 8. The benzyl protective group on the triethoxy chain was removed by hydrogenation to prepare alcohol 9. The nitrophenyl carbonate 10 was formed with bis(4-nitrophenyl)carbonate (Sigma-Aldrich). The nitrophenyl carbonate 10 was coupled with Paclitaxel (AK Scientific) in the presence of DMAP to prepare carbonate 11. The protective silyl groups were removed with tetrabutylammonium fluoride buffered in the presence of acetic acid to form the target product AB-1.

### Experimental Section:

Reagents were purchased from common commercial vendors including; Sigma-Aldrich, TCI America, and AK Scientific at the highest possible purity.

### 2,3,4,6-bis-acetonide-1-methyl-D-mannopyranoside 2

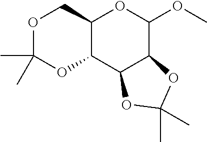

1-Methyl-D-mannopyranoside (4.0 g, 20.6 mmol) was mixed in DMF (16 mL) under an argon atmosphere at room temperature. 2,2-Dimethoxypropane (16 mL) was added at once followed by p-toluenesulfonic acid (100 mg). The solution stirred for 20 hours at room temperature under argon. Saturated sodium bicarbonate (30 mL) was added in portions followed by dichloromethane (100 mL). the dichloromethane layer was separated, washed with water, (50 mL), and dried over sodium sulfate. After filtration, the dichloromethane was removed under reduced pressure and the product was dried under high vacuum to a constant weight. The procedure prepared 2,3,4,6-bis-acetonide-1-methyl-D-mannopyranoside 2 (4.94 g, 88% yield) as a colorless solid. ¹H NMR (300 MHz, CDCl₃): 4.91 (s, 1H), 4.20-4.10 (m, 2H), 3.92-3.50 (m, 4H), 3.37 (s, 3H), 1.55 (s, 3H), 1.52 (s, 3H), 1.43 (s, 3H), 1.35 (s, 3H). ¹³C (75 MHz, CDCl₃): 109.54, 99.81, 99.00, 76.19, 75.07, 72.90, 62.29, 61.45, 55.17, 29.31, 28.43, 26.37, 19.06.

### 2,3-acetonide-1-methyl-D-mannopyranoside 3

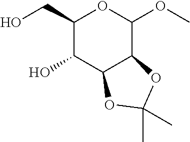 [SMILES: COC1O[C@H](CO)[C@@H](O)[C@@H]2OC(C)(C)O[C@H]12]

2,3,4,6-Bis-acetonide-1-methyl-D-mannopyranoside 2 (2.0 g, 7.29 mmol) was added to water/acetic acid (3:1, 20 mL) and was stirred at room temperature until the material completely dissolved (˜4 hours). Saturated potassium carbonate was added in portions until the pH=7. The product was extracted with dichloromethane (3×100 mL). The dichloromethane extracts were combined, dried over sodium sulfate, filtered, and concentrated. The procedure generated crude 2,3-acetonide-1-methyl-D-mannopyranoside 3 (1.7 g, 100% yield, 85% purity by NMR) that was used for the next step without purification. ¹H NMR (300 MHz, CDCl₃): 4.99 (s, 1H), 4.26-4.19 (m, 2H), 3.96-3.70 (m, 4H), 3.47 (s, 3H), 1.60 (s, 3H), 1.43 (s, 3H). ¹³C (75 MHz, CDCl₃): 109.75, 98.57, 78.55, 75.07, 68.84, 69.64, 62.62, 55.30, 28.21, 26.39.

### ((2-(2-(2-bromoethoxy)ethoxy)ethoxy)methyl)benzene 5

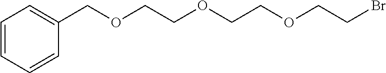 [SMILES: BrCCOCCOCCOCc1ccccc1]

2-(2-(2-(benzyloxy)ethoxy)ethoxy)ethanol (1.0 g, 4.16 mmol) and carbon tetrabromide (1.40 g, 4.2 mmol) were dissolved in dichloromethane under an argon atmosphere. The flask was placed in a water bath and triphenylphosphine (1.10 g, 4.2 mmol) in dichloromethane (10 mL) was added drop-wise over 15 minutes. After stirring for an additional 2 hours at room temperature, the solution was concentrated and the product was purified by flash column chromatography on silica gel (30 g), eluting with a mixture of heptanes/ethyl acetate (3:1). The experiment generated ((2-(2-(2-bromoethoxy)ethoxy)ethoxy)methyl)benzene 5 (1.0 g, 80% yield) as a colorless liquid. ¹H NMR (300 MHz, CDCl₃): 7.60-7.40 (m, 5H), 4.56 (s, 2H), 3.79 (t, 2H, J=6.3 Hz), 3.72-3.55 (m, 8H), 3.44 (t, 2H, J=6.3 Hz). ¹³C (75 MHz, CDCl₃): 138.24, 128.37, 127.74, 127.61, 73.34, 71.32, 70.83, 70.76, 70.66, 69.55, 30.55.

### (3aS,6R,7R,7aS)-4-methoxy-2,2-dimethyl-6-(12-phenyl-2,5,8,11-tetraoxadodecyl)tetrahydro-3aH-[1,3]dioxolo[4,5-c]pyran-7-ol 6

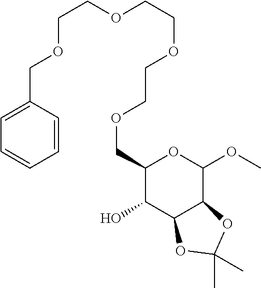 [SMILES: COC1O[C@H](COCCOCCOCCOCc2ccccc2)[C@@H](O)[C@@H]2OC(C)(C)O[C@H]12]

2,3-Acetonide-1-methyl-D-mannopyranoside 3 (1.2 g, 5.13 mmol) was dissolved in DMF (20 mL) at room temperature under an argon atmosphere with ((2-(2-(2-bromoethoxy)-ethoxy)ethoxy)methyl)benzene 5 (1.55 g, 5.13 mmol). Sodium hydride (205 mg, 60%, 5.13 mmol) was added and the mixture was slowly heated to 89-90° C. After 4 hours at 89-90° C., the heating was turned off and the mixture was allowed to cool to room temperature and stir overnight under an argon atmosphere. Water (50 mL) was added and the product was extracted twice with dichloromethane (100 mL). The combined dichloromethane extracts, were dried over sodium sulfate, filtered, concentrated, and dried under high vacuum overnight to remove DMF. The crude material was purified by flash column chromatography on silica gel (50 g), eluting with ethyl acetate. The experiment produced (3aS,6R,7R,7aS)-4-methoxy-2,2-dimethyl-6-(12-phenyl-2,5,8,11-tetraoxadodecyl)tetrahydro-3aH-[1,3]dioxolo[4,5-c]pyran-7-ol 6 (1.34 g, 57% yield) as a colorless oil. ¹H NMR (300 MHz, CDCl₃, major isomer): 7.40-7.20 (m, 5H), 4.91 (s, 1H), 4.57 (s, 2H), 4.25-3.85 (m, 3H), 3.80-3.50 (m, 15H), 3.36 (s, 3H), 2.81 (dd, 1H, J=7.5, 6.0 Hz), 1.54 (s, 3H), 1.35 (s, 3H). ¹³C (75 MHz, CDCl₃, major isomer): 138.32, 128.45, 127.86, 127.6, 109.41, 98.49, 78.97, 78.15, 76.05, 73.41, 70.83, 69.58, 68.45, 62.79, 55.11, 28.31, 26.55.

### (3S,4S,5S,6R)-6-(12-phenyl-2,5,8,11-tetraoxadodecyl)tetrahydro-2H-pyran-2,3,4,5-tetraol 7

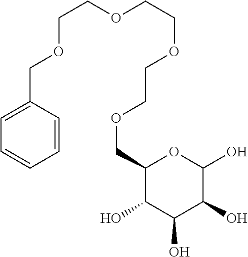 [SMILES: OC1O[C@H](COCCOCCOCCOCc2ccccc2)[C@@H](O)[C@H](O)[C@@H]1O]

(3aS,6R,7R,7aS)-4-methoxy-2,2-dimethyl-6-(12-phenyl-2,5,8,11-tetraoxadodecyl)tetrahydro-3aH-[1,3]dioxolo[4,5-c]pyran-7-ol 6 (1.30 g, 2.84 mmol) was heated to 60-65° C. in 20% hydrochloric acid for 48 hours. The material was concentrated under reduced pressure to prepare (3S,4S,5S,6R)-6-(12-phenyl-2,5,8,11-tetraoxadodecyl)tetrahydro-2H-pyran-2,3,4,5-tetraol 7 (1.15 g, 100% crude yield) as alight yellow oil. The material was used without purification for the next step. ¹H NMR (300 MHz, CD₃OD, major isomer): 7.45-7.20 (m, 5H), 5.15 (s, 1H), 4.57 (s, 2H), 4.0-3.5 (m, 18H). ¹³C (75 MHz, CD₃OD, major isomer): 139.47, 129.33, 128.84, 128.64, 95.64, 77.64, 74.10, 72.87, 72.39, 71.97, 71.50, 71.44, 70.60, 62.66.

### ((3S,4S,5R,6R)-6-(12-phenyl-2,5,8,11-tetraoxadodecyl)tetrahydro-2H-pyran-2,3,4,5-tetrayl)tetrakis(oxy)tetrakis(tert-butyldimethylsilane) 8

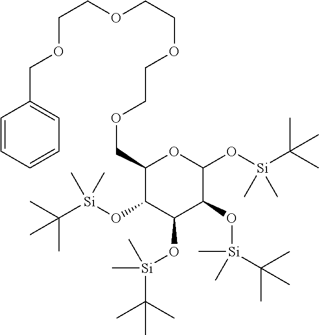 [SMILES: CC(C)(C)[Si](C)(C)OC1O[C@H](COCCOCCOCCOCc2ccccc2)[C@@H](O[Si](C)(C)C(C)(C)C)[C@H](O[Si](C)(C)C(C)(C)C)[C@@H]1O[Si](C)(C)C(C)(C)C]

(3S,4S,5S,6R)-6-(12-phenyl-2,5,8,11-tetraoxadodecyl)tetrahydro-2H-pyran-2,3,4,5-tetraol 7 (1.15 g, 2.86 mmol), t-butyldimethylsilylchloride (3.0 g, 20 mmol), imidazole (2.50 g, 36.0 mmol), and DMF (40 mL) were heated to 100-110° C. for 24 hours. The DMF was removed under high vacuum (at 30-40° C.). After cooling to room temperature, the remaining salts were extracted with heptane (2×100 mL). The combined heptanes extracts were filtered and concentrate. The crude product was purified by flash column chromatography on silica gel (100 g), eluting with a gradient of 100% heptanes to 1:1 heptane/ethyl acetate. Two main fractions were collected. The first contained ((3S,4S,5R,6R)-6-(12-phenyl-2,5,8,11-tetraoxadodecyl)-tetrahydro-2H-pyran-2,3,4,5-tetrayl)tetrakis(oxy)tetrakis(tert-butyldimethylsilane) 8 (0.70 g, 30% yield). The second fraction contained the tris-TBDMS protected (1 g, 40%) material that could be reprocessed (as above) to generate additional product. ¹H NMR (300 MHz, CDCl₃, major isomer): 7.40-7.20 (m, 5H), 4.57 (s, 2H), 4.48 (s, 1H), 3.90-3.40 (m, 18H), 1.0-0.80 (m, 36H), 0.20-0.0 (m, 24H). ¹³C (75 MHz, CDCl₃, major isomer): 138.38, 128.47, 127.86, 127.69, 101.55, 92.29, 73.73, 73.47, 73.10, 72.26, 70.92, 69.67, 62.88, 26.56, 26.30, 26.17, 26.00, 18.47 (m), −2.59, −3.69, −4.06, −4.74.

### 2-(2-(2-(((2R,3R,4S,5S)-3,4,5,6-tetrakis(tert-butyldimethylsilyloxy)tetrahydro-2H-pyran-2-yl)methoxy)ethoxy)ethoxy)ethanol 9

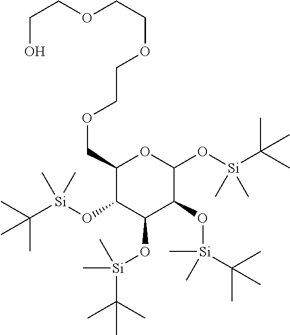 [SMILES: CC(C)(C)[Si](C)(C)OC1O[C@H](COCCOCCOCCO)[C@@H](O[Si](C)(C)C(C)(C)C)[C@H](O[Si](C)(C)C(C)(C)C)[C@@H]1O[Si](C)(C)C(C)(C)C]

((3S,4S,5R,6R)-6-(12-Phenyl-2,5,8,11-tetraoxadodecyl)-tetrahydro-2H-pyran-2,3,4,5-tetrayl)tetrakis(oxy)tetrakis(tert-butyldimethylsilane) 8 (0.65 g, 0.76 mmol) was dissolved in ethyl acetate (50 mL) and added to 10% palladium on carbon (0.70 g). The material was hydrogenated on a Parr apparatus at 50 psi of hydrogen for 2.5 hours at room temperature. After purging with nitrogen gas, the catalyst was removed by filtration through a pad of celite, washing with ethyl acetate (25 mL). The ethyl acetate solution was concentrated under reduced pressure. The experiment generated 2-(2-(2-(((2R,3R,4S,5S)-3,4,5,6-tetrakis(tert-butyldimethyl-silyloxy)tetrahydro-2H-pyran-2-yl)methoxy)ethoxy)ethoxy)ethanol 9 (0.54 g, 93% crude yield) as a colorless oil, which was used without purification for the next step. ¹H NMR (300 MHz, CDCl₃, major isomer): 4.90 (s, 1H), 4.20-3.50 (m, 18H), 1.0-0.80 (m, 36H), 0.20-0.0 (m, 24H). ¹³C (75 MHz, CDCl₃, major isomer): 95.48, 75.89, 75.43, 73.84, 72.76, 72.66, 72.14, 70.92, 70.24, 70.67, 62.90, 62.6, 26.58, 26.37, 26.18, 25.90, 18.51 (3 peaks), −3.84, −3.97, −4.14, −4.70, −5.00, −5.40.

### 4-nitrophenyl 2-(2-(2-(((2R,3R,4S,5S)-3,4,5,6-tetrakis(tert-butyldimethylsilyloxy)tetrahydro-2H-pyran-2-yl)methoxy)ethoxy)ethoxy)ethyl carbonate 10

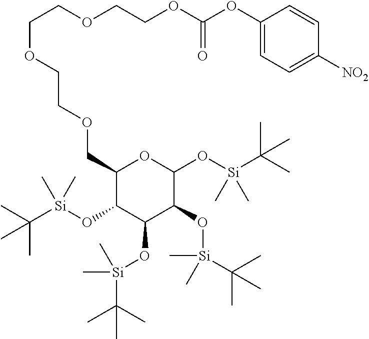 [SMILES: CC(C)(C)[Si](C)(C)OC1O[C@H](COCCOCCOCCOC(=O)Oc2ccc([N+](=O)[O-])cc2)[C@@H](O[Si](C)(C)C(C)(C)C)[C@H](O[Si](C)(C)C(C)(C)C)[C@@H]1O[Si](C)(C)C(C)(C)C]

2-(2-(2-(((2R,3R,4S,5S)-3,4,5,6-Tetrakis(tert-butyldimethyl-silyloxy)tetrahydro-2H-pyran-2-yl)methoxy)ethoxy)ethoxy)ethanol 9 (0.50 g, 0.64 mmol) was dissolved in dichloromethane (50 mL) and bis-nitrophenylcarbonate (5.0 g, 16.44 mmol) was added under an argon atmosphere at room temperature. Hunig's base (5.0 mL) was added drop-wise over 5 minutes. The mixture stirred for 40 hours under argon at room temperature. The mixture was concentrated under reduced pressure. Dichloromethane (20 mL) was added to the yellow solid followed by heptane (100 mL). After stirring for 2 hours at room temperature, the solvent was filtered to remove unreacted starting material. The filtrate was concentrated and the product purified by flash column chromatography on silica gel (25 g), eluting with 100% heptanes to 30% ethyl acetate in heptanes. The product was collected in two isomer fractions. The experiment produced 4-nitrophenyl 2-(2-(2-(((2R,3R,4S,5S)-3,4,5,6-tetrakis(tert-butyldimethylsilyloxy)tetrahydro-2H-pyran-2-yl)methoxy)ethoxy)ethoxy)ethyl carbonate 10 (0.40 g major isomer, 0.08 g minor isomer, 80% combined yield) as a clear oil. ¹H NMR (300 MHz, CDCl₃, major isomer): 8.37 (d, 2H, J=9 Hz), 7.48 (d, 2H, J=9 Hz), 4.99 (d, 1H, J=1.8 Hz), 4.52 (m, 2H), 4.20-3.60 (m, 16H), 1.05-0.90 (m, 36H), 0.22-0.0 (m, 24H). ¹³C (75 MHz, CDCl₃, major isomer): 155.73, 152.62, 145.63, 125.42, 121.88, 95.58, 77.44, 76.06, 75.57, 74.05, 72.91, 72.22, 71.05, 70.85, 68.96, 68.63, 63.05, 26.62, 26.41, 26.23, 25.94, 18.78, 18.57 (2 peaks), 18.25, −3.81, −3.93, −4.10, −4.68, −4.96, −5.34.

### Paclitaxel-Triethoxy-TBDMS-Mannose 11:

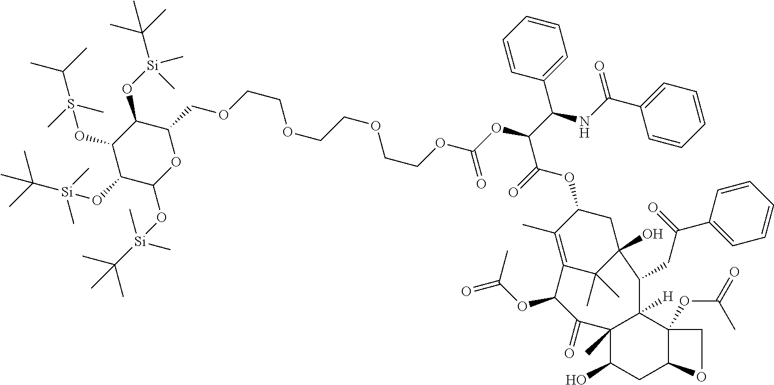 [SMILES: CC(=O)O[C@@H]1C(=O)[C@@]2(C)[C@H](O)C[C@@H]3OC[C@]3(OC(C)=O)[C@@H]2[C@@H](CC(=O)c2ccccc2)[C@@]2(O)C[C@@H](OC(=O)[C@@H](OC(=O)OCCOCCOCCOC[C@@H]3OC(O[Si](C)(C)C(C)(C)C)[C@H](O[Si](C)(C)C(C)(C)C)[C@H](OS(C)(C)C(C)C)[C@H]3O[Si](C)(C)C(C)(C)C)[C@H](NC(=O)c3ccccc3)c3ccccc3)C(C)=C1C2(C)C]

Paclitaxel (240 mg, 0.28 mmol), DMAP (50 mg, 0.41 mmol), and 24242-(((2R,3R,4S,5S)-3,4,5,6-tetrakis(tert-butyldimethylsilyloxy)tetrahydro-2H-pyran-2-yl)methoxy)ethoxy)-ethoxy)ethyl carbonate 10 (290 mg major isomer, 0.31 mmol) were dissolved in dichloromethane (5 mL) under an argon atmosphere at room temperature. The solution stirred for 24 hours at room temperature. The solution was concentrated and purified by flash column chromatography on silica gel (10 g), eluting with heptanes to 40% ethyl acetate in heptanes. The experiment generated Paclitaxel-triethoxy-TBDMS-mannose 11 (400 mg, 78% yield) as a white solid glass.

¹H NMR (300 MHz, CDCl₃): 8.13 (d, J=7 Hz, 2H), 7.74 (d, J=7 Hz, 2H), 7.62-7.20 (m, 12H), 6.95 (d, 1H, J=9.3 Hz), 6.28 (m, 2H), 5.98 (d, J=9.6 Hz, 1H), 5.68 (d, J=6.9 Hz, 1H), 5.40 (d, 1H, J=2.4 Hz), 5.00-4.85 (m, 2H), 4.60-4.10 (m, 4H), 4.05-3.45 (m, 19H), 2.75-2.46 (m, 2H), 2.45 (s, 3H), 2.45-2.32 (m, 1H), 2.22 (s, 3H), 1.94 (s, 3H), 1.68 (s, 3H), 1.23 (s, 3H), 1.14 (s, 3H), 0.95-0.80 (m, 36H), 0.10-0.10 (m, 24H). ¹³C NMR (75 MHz, CDCl₃/TMS): 203.81, 171.31, 169.92, 167.88, 167.23 (2 peaks), 154.26, 142.81, 136.86, 133.79, 132.93, 132.15, 130.04, 129.23 (2 peaks), 128.86, 128.83, 128.61, 127.31, 126.68, 95.48, 84.65, 81.29, 79.36, 76.99, 75.82 (2 peaks), 75.41 (2 peaks), 73.84, 72.25 (3 peaks), 70.92, 70.84, 70.66, 68.92, 68.35, 62.89, 58.77, 52.91, 45.82, 43.48, 35.91 (2 peaks), 27.15, 26.57, 26.36, 26.17, 25.89, 23.01, 22.51, 21.13, 18.73, 18.51 (2 peaks), 18.19, 15.09, 9.94, −3.85, −3.98, −4.15, −4.70, −5.00, −5.42.

### Paclitaxel-Triethoxy-Mannose Analog AB-1:

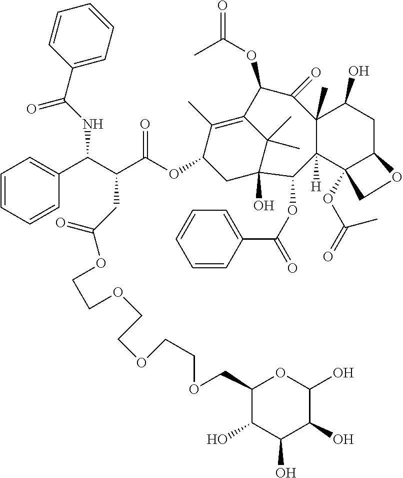 [SMILES: CC(=O)O[C@H]1C(=O)[C@@]2(C)[C@H]([C@H](OC(=O)c3ccccc3)[C@]3(O)C[C@H](OC(=O)[C@H](CC(=O)OCCOCCOCCOC[C@H]4OC(O)[C@@H](O)[C@@H](O)[C@@H]4O)[C@@H](NC(=O)c4ccccc4)c4ccccc4)C(C)=C1C3(C)C)[C@]1(OC(C)=O)CO[C@@H]1C[C@@H]2O]

General Procedure: Paclitaxel-triethoxy-TBDMS-mannose 11 (400 mg, 0.12 mmol) was converted in batches (50-200 mg each) to the unprotected product. A batch of Paclitaxel-triethoxy-TBDMS-mannose 11 was dissolved in a small volume of THF (1-2 mL) under an argon atmosphere. Acetic acid (40 equivalents) was added followed by tetrabutylammonium fluoride (30 equivalents). The solution stirred for 5 days at room temperature under argon. The reaction was usually 50% complete after 5 days. The THF was removed under reduced pressure and dichloromethane (50 mL) was added. The bulk of the salts were removed by extraction with water (2×20 mL). The dichloromethane was dried over sodium sulfate, filtered and concentrated. The product was separated from partially protected material (mostly bis-TBDMS) by flash column chromatography on silica gel, eluting with 5% methanol in dichloromethane. The product was set aside and the partially protected material was reprocessed as above. Once all of the Paclitaxel-triethoxy-TBDMS-mannose 11 (400 mg) was converted and the reprocessed, the combined product were repurified by column chromatography on silica gel, eluting with 5% methanol in dichloromethane. The product containing fractions were combined and concentrated. The residue was dissolved in a small amount of dichloromethane and triturated into diethyl ether. The precipitate was filtered and dried to a constant weight under high vacuum at room temperature. The experiments generated a pure Paclitaxel-triethoxy-mannose analog AB-1 (110 mg, 40% yield, 96.3% purity by HPLC) fraction as well as a slightly less pure filtrate (25 mg) fraction and a small amount of partially protected material (25 mg) that could be reprocessed.

### Analysis:

Appearance: White solid glass

Chemical Formula: C₆₀H₇₃NO₂₄

Molecular Weight: 1192.21

HRMS: Calculated for [M+Na] 1214.4415

Found: 1214.4446

Chromatographic purity (HPLC): 96.3%

¹H NMR (300 MHz, CDCl₃/TMS): δ 8.14 (d, J=7.5 Hz, 2H), 7.77 (d, J=7.8 Hz, 2H), 7.64-7.20 (m, 12H), 6.32-6.20 (m, 2H), 6.00 (m, 1H), 5.69 (d, J=7.2 Hz, 1H), 5.45 (d, J=2.1 Hz, 1H), 4.97 (m, 2H), 4.70-4.17 (m, 5H), 3.95-3.10 (m, 19H), 2.75-2.46 (m, 2H), 2.46 (s, 3H), 2.45-2.32 (m, 1H), 2.23 (s, 3H), 1.94 (s, 3H), 1.68 (s, 3H), 1.23 (s, 3H), 1.14 (s, 3H).

¹³C NMR (75 MHz, CDCl₃/TMS): δ 203.70, 171.22, 170.21, 170.09, 168.13, 167.40 (d), 167.12, 154.35, 142.68 (d), 137.01 (d), 134.04 (d), 133.76, 133.14 (d), 132.01 (d), 130.40, 129.54, 129.22 (d), 128.89, 128.78 (d), 128.58 (d), 127.49 (d), 127.43, 126.90, 94.38, 84.72, 81.52, 79.45, 77.98, 77.43, 75.91, 75.47, 74.13, 72.52, 72.37, 72.28, 72.13, 71.93, 71.60, 71.39, 71.18, 70.69, 70.53, 68.95, 68.38, 62.31, 61.99, 58.86, 53.16, 53.03, 46.07, 43.57, 36.03, 27.19, 23.02, 22.48, 21.12, 15.09, 10.02. * The doubling in C13 spectrum is likely due to rotational isomerism caused by the carbamate bridge.

### REFERENCES

1. Scott A M, et al., Monoclonal antibodies in cancer therapy. Cancer Immun. 2012; 12:14. PMID 22896759. Epub 2012 May 1.
2. Blasi, P., Solid lipid nanoparticles for targeted brain drug delivery. Science Direct. 2007; 454-477.
   HPB (Oxford). 10(1): 58-62. Doi: 10.1080/1365182070188314.
3. Georgieva J V, et al., Smuggling drugs into the brain: An overview of ligands targeting transcytosis for drug delivery across the blood-brain barrier.

Pharmaceutics. 2014; 6:557-83). PMID 25407801. Doi: 10.3390/pharmaceutics6040557.
4. Sarvaiya, J., et al., Chitosan as a suitable nanocarrier material for anti-Alzheimer drug delivery. Int J Biol Macromol. 2015; 72:454-65. Doi: 10.1016/j.ijbiomac.2014.08.052. PMID 25199867. Epub 2014 Sep. 6.
5. Blanco E, et al., Nanomedicine in cancer therapy: innovative trends and prospects. Cancer Sci. 2011; 102:1247-52. Doi: 10.1111/j.1349-7006.2011.01941.x. PMID 21447010. Epub 2011 May 3.
6. Gowda, R. et al., Use of Nanotechnology to Develop Multi-Drug Inhibitors for Cancer therapy. J Nanomed Nanotech. 2014; 4(6): .doi: 10.4172/2157-7439.1000184.
7. Mohanty C, et al., Receptor mediated tumor targeting: an emerging approach for cancer therapy. Curr Drug Deliv 2011; 8:45-58. PMID 21034422.
8. Guo, A., et al., Signaling networks assembled by oncogenic EGFR and c-Met. Proc Natl Acad Sci USA 2008; 105:692-7. Doi: 10.1073/pnas.0707270105. PMID 18180459. Epub 2008 Jan. 7.
9. Sierra J R, et al., c-Met as a potential therapeutic target and biomarker in cancer. Ther Adv Med Oncol. 2011 November; 3(1 Suppl):521-35. doi: 10.1177/1758834011422557. PMID 22128285.
10. Perez-Herrero, E., et al., Advanced targeted therapies in cancer: Drug nanocarriers, the future of chemotherapy. Eur j Pharm Biopharm 2015 Mar. 23; pii: S0939-6411(15)00151-4 doi: 10.1016/j.ejpb.2015.03.018. [Epub ahead of print].
11. Jain, R. K., et al., Delivering nanomedicine to solid tumors. Nature Reviews Clin. Oncol. 2010; 7:653-664.
12. Reddy, L. H., et al., Novel approaches to deliver gemcitabine to cancers. Curr Pharm Des. 14(11):1124-1137; 2008.
13. Federico, C., et al., Gemcitabine-loaded liposomes: rationale, potentialities, & future perspectives. Int. J Nanomedicine. 2012; 7:5423-36.
14. Paolino, D. et al., Gemcitabine-loaded PEGylated unilamellar liposomes vs Gemzar®: Biodistribution, pharmacokinetic features and in vivo antitumor activity. 2010; Journal of Controlled Release. 144:144-150. Doi: 10.1016/j.conrel.2010.02.021
15. Brusa, P., et al., Antibody activity and pharmacokinetics of liposome containing lipophilic gemcitabine prodrugs. Anticancer Res. 2007; 27(1A):195-199.
16. Noh, I., et al., Co-delivery of paclitaxel and gemcitabine via CD44-targeting nanocarriers as a prodrug with synergistic antitumor activity against human biliary cancer. Biomaterials. June 2015; 53:763-774.
17. Cosco, D., et al., Liposomes as multicompartmental carriers for multidrug delivery in anticancer chemotherapy. Drug Deliv Transl Res. 2011; 1 (1): 66-75.
18. U. Le, et al., Liposome Formulation of Gemcitabine and Curcumin for Cancer Treatment. Abstracts.aaps.org/verify/aaps2013/postersubmissions/W4093.pdf
19. Tan, M., et al., Delivering curcumin and gemcitabine in one nanoparticle platform for colon cancer. RSC Adv. 2014; 4, 61948-61959.
20. Xu, H., et al., Development of gradient high performance liquid chromatography assay for simultaneous analysis of hydrophilic gemcitabine and lipophilic curcumin using a central composite design and its application in liposome development. J Pharm Biomed Anal. 2014 September; 98:371-8. Doi: 10.1016/j.jpba.2014.06.022. Epub 2014 Jun. 21.
21. Aryal, S., et al., Combinatorial drug conjugation enables nanoparticle dual-drug delivery. Small. 6:1442-1448; 2010.
22. Lanni, J., et al., P53-Independent apoptosis induced by paclitaxel through an independent mechanism. PNAS. 94(18): 9679-9683; 1997
23. Shi, Jin-Yuan, et al. Tumor-associated macrophage: Its role in cancer invasion and metastasis. J. of Cancer Molecules. 71(5): 1825-1835; 2011.
24. Kan, P., et al., A Liposomal Formulation Able to Incorporate a High Content of Paclitaxel and Exert Promising Anticancer Effect. Journal of Drug Delivery. Volume 2011 (2011), Article ID 629234, 9 pages. http://dx.doi.org/10.1155/2011/629234
25. Fan, Y., et al., Development of liposomal formulations: From concept to clinical investigations. Asian J of Pharm Sciences. 2013; 8:81-87.
26. Koudelka, S., et al., Liposomal paclitaxel formulations. J Control Release. 2012 Nov. 10; 163(3):322-34. Doi: 10.1016/j.conrel.2012.09.006. Epub 2012 Sep. 15.
27. Li, Lan., M. D., et al., Liposome-encapsulated curcumin: in vitro and in vivo effects on proliferation, apoptosis, signaling, and angiogenesis. Cancer. 2005; 104(6):1322-1331.
28. Bisht, S., et al., Systemic administration of polymeric nanoparticle-encapsulated curcumin (NanoCurc) blocks tumor growth and metastases in preclinical models of pancreatic cancer. Mol Cancer Ther. 2010; 9:2255-2264.
29. Kim, T. H., et al., Preparation and characterization of water-soluble albumin-bound curcumin nanoparticles with improved antitumor activity. Int J Pharm. 2011; 403:286-91.
30. Wang, D., et al., Liposome-encapsulated curcumin suppresses growth of head and neck squamous cell carcinoma in vitro and in xenografts through inhibition of nuclear factor kappaB, an AKT-independent pathway. Clin Cancer Res. 2008; 14:6228-36.
31. Shi, H. S., et al., A systemic administration of liposomal curcumin inhibits radiation pneumonitis and sensitizes lung carcinoma to radiation. Int J Nanomedicine. 2012; 7:2601-11.
32. Duarte, V. M., et al., curcumin enhances the effect of cisplatin in suppression of head and neck squamous cell carcinoma via inhibition of IKKbeta protein of the NFkappaB pathway. Mol Cancer Ther. 2010; 9:2665-75.
33. Aggarwal, S., et al., [DLys (6)]-luteinizing hormone releasing hormone-curcumin conjugate inhibits pancreatic cancer cell growth in vitro and in vivo. Int J Cancer. 2011; 129:1611-23.
34. Gou, M., et al., Curcumin-loaded biodegradable polymeric micelles for colon cancer therapy in vitro and in vivo. Nanoscale. 2011; 3:1558-67.
35. Kumar, A., et al., Curcumin loaded nanoglobules for solubility enhancement: preparation, characterization and ex vivo release study. J Nanosci Nanotechnol. 2012; 12:8293-302.
36. Guzman-Villanueva, D., et al., Design and in vitro evaluation of a new nano-microparticulate system for enhanced aqueous-phase solubility of curcumin. Biomed Res Int. 2013; 724-763.
37. Zhongfa, L., et al., Enhancement of curcumin oral absorption and pharmacokinetics of curcuminoids and curcumin metabolites in mice. Cancer Chemother Pharmacol. 2012; 69:679-89.
38. Gandapu, U., et al., Curcumin-loaded apotransferrin nanoparticles provide efficient cellular uptake and effectively inhibit HIV-1 replication in vitro. PLoS One. 2011; 6:e23388.
39. Cheng, K K, et al., Highly stabilized curcumin nanoparticles tested in an vitro blood blood-brain barrier model and in Alzheimer's disease Tg2576 mice. AAPS J. 2013; 15:324-36.
40. Anand, P., et al., Design of curcumin-loaded PLGA nanoparticles formulation with enhanced cellular uptake, and increased bioactivity in vitro and superior bioavailability in vivo. Biochem Pharmacol. 2010; 79:330-8.
41. Khalil, N. M., et al., Pharmacokinetics of curcumin-loaded PLGA and PLGA-PEG blend nanoparticles after oral administration in rats. Colloids Surf B Biointerfaces. 2013; 101:353-60.
42. Tsai, Y. M., et al., Effects of polymer molecular weight on relative oral bioavailability of curcumin. Int J Nanomedicine. 2012; 7:2957-66.
43. Xie, X., et al., PLGA nanoparticles improve the oral availability of curcumin in rats: characterizations and mechanisms. J Agric Food Chem. 2011; 59:9280-9.
44. Yallapu, M. M., et al., Fabrication of curcumin encapsulated PLGA nanoparticles for improved therapeutic effects in metastatic cancer cells. J Colloid Interface Sci. 2010; 351:19-29.
45. Takahashi, M., et al., Evaluation of an oral carrier system in rats: bioavailability and antioxidant properties of liposome-encapsulated curcumin. J Agric Food chem. 2009; 57:9141-6.
46. Li, Lan., M. D., et al., Liposome-encapsulated curcumin: in vitro and in vivo effects on proliferation, apoptosis, signaling, and angiogenesis. Cancer. 2005; 104(6):1322-1331.
47. Li, C., et al., Silica-coated flexible liposomes as a nanohybrid delivery system for enhanced oral bioavailability of curcumin. Int J Nanomedicine. 2012; 7:5995-6002.
48. Chen, H., et al., N-trimethyl chitosan chloride-coated liposomes for the oral delivery of curcumin. J Liposome Res. 2012; 22:100-9.
49. Zhang, L., Physical characterization and cellular uptake of propylene glycol liposomes in vitro. Drug Dev Ind Pharm. 2012; 38:365-71.
50. Yadav, V. R., et al., Cyclodextrin-complexed curcumin exhibits anti-inflammatory and antiproliferative activities superior to those of curcumin through higher cellular uptake. Biochem Pharmacol. 2010; 80:1021-32.
51. Rachmawati, H., et al., Molecular inclusion complex of curcumin-beta-Cyclodextrin nanoparticle to enhance curcumin skin permeability from hydrophilic matrix gel.
AAPS Pharm SciTech. 2013; 14:1303-12.
52. Shoba, G. et al., Influence of piperine on the pharmacokinetics of curcumin in animals and human volunteers. Planta Med. 1998; 64:353-6.
53. Thangapazham, R. L., et al., Evaluation of nanotechnology-based carrier for delivery of curcumin in prostate cancer cells. Int J Oncol. 2008; 32:1119-23.
54. Ghosh, D., et al., Nanocapsulated curcumin: oral chemopreventive formulation against diethylnitrosamine induced hepatocellular carcinoma in rat. Chem Biol Interact. 2012; 195:206-14.
55. Misra, R. et al., Coformulation of doxorubicin and curcumin in poly (D, L-lactide-co-glycolide) nanoparticles suppresses the development of multidrug resistance in K562 cells. Mol Pharm. 2011; 8:852-66.
56. Agashe, H., et al., Cyclodextrin-mediated entrapment of curcuminoid 4-[3, 5-bis (2-chlorbenzylidine-4-oxo-piperidine-1yl)-4-oxo-2-butenoic acid] or CLEFMA in liposomes for treatment of xenograft lung tumor in rats. Colloids Surf B Biointerfaces. 2011; 84:329-37.
57. Alam, S., et al., Novel dipeptide nanoparticles for effective curcumin delivery. Int J Nanomedicine. 2012; 7:4207-22.
58. Aditya N. P., et al., Curcuminoids-loaded liposomes in combination with arteether protects against Plasmodium berghei infection in mice. Exp Parasitol. 2012; 131:292-9.
59. Basnet, P., et al., Liposomal delivery system enhances anti-inflammatory properties of curcumin. J Pharm Sci. 2012; 101:598-609.
60. Pandelidou, M., et al., Preparation and characterization of lyophilized egg PC liposomes incorporating curcumin and evaluation of its activity against colorectal cancer cell lines. J Nanosci Nanotechnol. 2011; 11:1′259-66.
61. Mourtas, S., et al., Curcumin-decorated nanoliposomes with very high affinity for amyloid-beta 1-42 peptide. Biomaterials. 2011; 32: 1635-45.
62. Luo, F., et al., Low-dose curcumin leads to inhibition of tumor growth via enhancing CTL-mediated antitumor immunity. Int Immunopharmacol. 11(9): 1234-40; 2011.
63. Hayakawa, T., et al., Enhanced anti-tumor effects of the PD-1/PD-L1 blockade by combining a highly absorptive form of NF-kB/STAT3 inhibitor curcumin. Journal for Immunotherapy of Cancer 2(Suppl 3): P210 doi:10.1186/2051-1426-2-S3-P210; 2014
64. Cao, J., et al., Curcumin induces apoptosis through mitochondrial hyperpolarization and mtDNA damage in human hepatoma G2 cells. Free radical biology and Medicine. 43: 968-975; 2007.
65. Shishodia, S., et al., Curcumin inhibits constitutive NF-kappa B activation, induces G1/S arrest, suppresses proliferation, and induces apoptosis in mantle cell lymphoma. Biochem Pharmacol. 70(5):700-13; 2005.
66. Bush, Jason A., et al., Curcumin induces apoptosis in human melanoma cells through a Fas Receptor/Caspase-8 pathway independent of p53. Exp Cell Res. 271 (2): 305-314; 2001.
67. Mohan, R., et al., Curcuminoids inhibit the angiogenic response stimulated by fibroblast growth factor-2, including expression of matrix metalloproteinase gelatinase B. J Biol Chem. 275(14):10405-12; 2000.
68. Anto, R. J., et al., Curcumin induces apoptosis through activation of caspase-8, BID cleavage and cytochrome c release; its suppression by ectopic expression of Bcl-2 and Bcl-xl. Carcinogenesis. 23(1):143-150; 2002.
69. Li, L., Aggarwal, B B, Nuclear factor kappa B and 1 kappa B kinase are constitutively active in pancreatic cells, and their down-regulation by curcumin is associated with suppression of proliferation and induction of apoptosis. Cancer. 101(10):2351-62; 2004.
70. Singh, S., et al., Activation of transcription factor NF-kappa B is suppressed by curcumin. J Biol Chem. 270(42):24995-5000; 1995.
71. Bharti, A. C., et al., Curcumin down-regulates the constitutive activation of nuclear factor kappa B and I kappa B alpha kinase in human multiple myeloma cells, leading to suppression of proliferation and induction of apoptosis. Blood. 101(3):1053-62; 2003.
72. Li, Lan., M. D., et al., Liposome-encapsulated curcumin: in vitro and in vivo effects on proliferation, apoptosis, signaling, and angiogenesis. Cancer. 104(6):1322-1331; 2005.
73. Kunnumakkara, A. B., et al., Curcumin potentiates antitumor activity of gemcitabine in an orthotopic model of pancreatic cancer through suppression of proliferation, angiogenesis, and inhibition of NF-kB-regulated gene products. Cancer Res. 67(8):3853-61; 2007.
74. Ali, S., et al. Gemcitabine sensitivity can be induced in pancreatic cancer cells through modulation of miR-200 and mir-21 expression by curcumin or its analogue CDF. Cancer Res. 70(9): 3606-17; 2010.
75. Bimonte, S., et al. Curcumin inhibits tumor growth and angiogenesis in an orthotopic mouse model of human pancreatic cancer. Biomed Res Int. 810423. Doi: 10.1155/2013/810423; Nov. 10, 2013.
76. Aggarwal, S., et al., Inhibition of growth and survival of human head and neck squamous cell carcinoma cells by curcumin via modulation of NF-kB signaling. Int J Cancer. 111(5):679-92; 2004.
77. Ono, K., et al., Curcumin has potent anti-amyloidogenic effects for Alzheimer's β-amyloid fibrils in vitro. J of Neuroscience Research. 15 Mar. 2004; 75:742-750. Doi: 1002/jnr.20025.
78. U. Massing, S. Cicko, V. Ziroli, Dual asymmetric centrifugation (DAC)—A new technique for liposome preparation, Journal of Controlled Release. 125 (2008) 16-24. doi:10.1016/j.jconrel.2007.09.010.
79. R. Moog, A. Burger, M. Brandl, J. Schüler, R. Schubert, C. Unger, et al., Change in pharmacokinetic and pharmacodynamic behavior of gemcitabine in human tumor xenografts upon entrapment in vesicular phospholipid gels, Cancer Chemother Pharmacol. 49 (2002) 356-366. doi:10.1007/s00280-002-0428-4.
80. Namdi, A.; Chattopadhyay, P.; Synthesis of chiral trans-fused pyrano[3,2-c][2]benzoxocines from D-mannose by regioselective 8-endo-aryl radical cyclization. Tetrahedron Letters 43 (2002) 5977-5980.
81. Š. Koudelka, J. Turánek, Liposomal paclitaxel formulations, Journal of Controlled Release. 163 (2012) 322-334. doi:10.1016/j.jconrel.2012.09.006.
82. U. Massing, Herstellung Von Lipidbasierten Nanopartikeln Unter Einsatz Einer Dualen Asymmetrischen Zentrifuge, WO2006069985A2
83. B. B. Aggarwal, R. Kurzrock, L. Li, K. Mehta, Liposomal Curcumin for Treatment of Neurofibromatosis, US20080103213A1
84. C. M. Mach, L. Mathew, S. A. Mosley, R. Kurzrock, J. A. Smith, Determination of Minimum Effective Dose and Optimal Dosing Schedule for Liposomal Curcumin in a Xenograft Human Pancreatic Cancer Model, Anticancer Res. 29 (2009) 1895-1899.
85. T. Yang, F.-D. Cui, M.-K. Choi, H. Lin, S.-J. Chung, C.-K. Shim, et al., Liposome Formulation of Paclitaxel with Enhanced Solubility and Stability, Drug Delivery. 14 (2007) 301-308. doi:10.1080/10717540601098799.

1. A method of preferentially delivering a pharmaceutical agent selected from the group consisting of gemcitabine, paclitaxel and/or curcumin intracellularly to myeloid antigen-presenting cells of a mammalian subject by using a nanocarrier comprising a Fucose-derivative anchor on its surface and having the pharmaceutical agent encapsulated in order to therapeutically address a malignancy or a neurodegenerative disorder.

2. The method of claim 1, wherein said myeloid antigen-presenting cell is selected from the group consisting of myeloid dendritic cells, cytotoxic T lymphocytes, circulating monocytes, macrophages, and tumor-associated macrophages.

3. The method of claim 1, wherein the nanocarrier is a lipid-based nanocarrier or a non-lipid based nanocarrier.

4. The method of claim 3, wherein the lipid-based nanocarrier is a liposome, a lipoplex, or a micelle.

5. The method of claim 1, wherein the malignancy includes, but is not limited to metastatic pancreatic adenocarcinoma, triple-negative breast cancer, small cell lung carcinoma, malignant melanoma, head and neck squamous cell carcinoma, renal cell carcinoma, prostate cancer, bladder cancer, small and large bowel carcinoma, thyroid carcinoma, non-Hodgkin's lymphoma, the leukemias, cervical carcinoma, ovarian carcinoma, Kaposi's sarcoma, osteosarcoma, basal cell carcinoma, and squamous cell carcinoma.

6. The method of claim 1, wherein the neurodegenerative disorder includes, but is not limited to Alzheimer's disease, Parkinson disease, spinal cord trauma, stroke, and multiple sclerosis.

7. The method of claim 1, wherein the nanocarrier-encapsulated pharmaceutical agent is delivered via an intravenous, a subcutaneous, an intratumoral, an intrametastatic, an intradermal, an intraperitoneal, a transdermal, a parenteral, or an intrapulmonary route, a route by infusion via the hepatic artery, an intrathyroidal route, an intranasal route, an intrathecal route, or a topical route.

8. The method of claim 7, wherein the mode of administration is parenterally.

9. The method of claim 1, wherein the combination of different permutations of the nanocarriers loaded with different pharmaceutical agents is to achieve an optimal therapeutic effect that is superior to the outcome that can be achieved with the non-encapsulated agents or the same pharmaceutical agents when encapsulated in non-targeted nanocarriers.

10. The method of claim 1, wherein the Fucose-derivative anchor is Fucose-4-chol.

11. A nanocarrier comprising a fucose-derivative anchor on its surface and having encapsulated a pharmaceutical agent selected from the group consisting of gemcitabine, paclitaxel and/or curcumin for use in a method to therapeutically address a malignancy or a neurodegenerative disorder in a mammalian subject, wherein the nanocarrier is delivered intracellularly to myeloid antigen-presenting cells of the mammalian subject.

12. The nanocarrier for the use of claim 11, wherein said myeloid antigen-presenting cell is selected from the group consisting of myeloid dendritic cells, cytotoxic T lymphocytes, circulating monocytes, macrophages, and tumor-associated macrophages.

13. The nanocarrier for the use of claim 11, wherein the nanocarrier is a lipid-based nanocarrier or a non-lipid based nanocarrier.

14. The nanocarrier for the use of claim 13, wherein the lipid-based nanocarrier is a liposome, a lipoplex, or a micelle.

15. The nanocarrier for the use of claim 11, wherein the malignancy includes, but is not limited to metastatic pancreatic adenocarcinoma, triple-negative breast cancer, small cell lung carcinoma, malignant melanoma, head and neck squamous cell carcinoma, renal cell carcinoma, prostate cancer, bladder cancer, small and large bowel carcinoma, thyroid carcinoma, non-Hodgkin's lymphoma, the leukemias, cervical carcinoma, ovarian carcinoma, Kaposi's sarcoma, osteosarcoma, basal cell carcinoma, and squamous cell carcinoma.

16. The nanocarrier for the use of claim 11, wherein the neurodegenerative disorder includes, but is not limited to Alzheimer's disease, Parkinson disease, spinal cord trauma, stroke, and multiple sclerosis.

17. The nanocarrier of claim 11, wherein the nanocarrier-encapsulated pharmaceutical agent is delivered via an intravenous, a subcutaneous, an intratumoral, an intrametastatic, an intradermal, an intraperitoneal, a transdermal, a parenteral, or an intrapulmonary route, a route by infusion via the hepatic artery, an intrathyroidal route, an intranasal route, an intrathecal route, or a topical route.

18. The nanocarrier for use of claim 17, wherein the mode of administration is parenterally.

19. The nanocarrier for the use of claim 11, wherein the combination of different permutations of the nanocarriers loaded with different pharmaceutical agents is to achieve an optimal therapeutic effect that is superior to the outcome that can be achieved with the non-encapsulated agents or the same pharmaceutical agents when encapsulated in non-targeted nanocarriers.

20. The nanocarrier for the use of claim 11, wherein the Fucose-derivative anchor is Fucose-4-chol.
